(19) United States
(12) Patent Application Publication
(10) Pub. No.: US 2026/0114830 A1
(43) Pub. Date: Apr. 30, 2026

(54) METHOD AND SYSTEM FOR DETERMINING HEMODYNAMIC PARAMETERS

(71) Applicant: Opsens Inc., Quebec (CA)

(72) Inventors: Frédéric COUET, Quebec (CA); Sébastien LALANCETTE, Saint-Augustin-de-Desmaures (CA); Stewart MCLENNAN, Verdun (CA)

(21) Appl. No.: 19/163,065

(22) PCT Filed: Mar. 9, 2023
(86) PCT No.: PCT/CA2023/050315

§ 371 Date: Sep. 8, 2025

(51) Int. Cl. A61B 6/50; A61B 5/00; A61B 5/0215; A61B 6/00; A61B 6/03; A61B 6/46; G06T 7/246; G06T 7/33; G16H 50/20

(52) U.S. Cl. CPC A61B 6/507; A61B 5/0215; A61B 5/4848; A61B 5/742; A61B 6/032; A61B 6/463; A61B 6/466; A61B 6/504; A61B 6/5205; G06T 7/251; G06T 7/344; G16H 50/20; A61B 2560/0238; G06T 2207/30104

(57) ABSTRACT

A method executable by a system comprising a processor in communication with an extraluminal imaging device and an intravascular pressure measurement device. The method comprises acquiring contrast-agent angiographic images in a blood vessel having a contrast agent; based on contrast-agent angiographic images, generating a reconstructed geometry object of the blood vessel and estimating a blood flow in the blood vessel; determining output parameters based on the values of the blood pressure and the blood flow, by implementing a physical model of blood distribution using the reconstructed geometry object; and generating a reconciled hemodynamic parameter based on the output parameters. The reconciled hemodynamic parameter is adjusted based on measurements performed at various blood vessel states.

### TECHNICAL FIELD

The present disclosure relates to methods and systems for assessment of hemodynamic function. More specifically, it relates to methods and systems for determining hemodynamic parameters using an intravascular pressure measurement device combined with medical imaging.

### BACKGROUND

Coronary artery diseases may be diagnosed by assessing an impact of coronary stenosis on blood flow. Coronary stenosis is related to thickening and narrowing of the coronary arteries and may occur in coronary arteries which are blood vessels that bring blood to the heart. Coronary stenosis may occur both in thick and narrow coronary arteries and can lead to angina which manifests in patients as a chest pain.

To determine and characterise the coronary stenosis, various hemodynamic parameters (sometimes also referred to as “hemodynamic quantities”) may be used. For example, a fractional flow reserve (FFR) determines a ratio between the maximum achievable blood flow through a blockage (area of stenosis) of a given coronary artery and a theoretical maximum flow in the same coronary artery in the hypothetical absence of the blockage. Another hemodynamic parameter that could help with the assessment of the state of the coronary artery is coronary flow reserve (CFR). The CFR value provides an estimation of the blood flow in epicardial vessels and microvasculature altogether.

An index of microvascular resistance (IMR) is a hemodynamic parameter that reflects coronary microvascular functions which relates to smallest blood vessels. Estimating the IMR may help to determine the characteristics of the blood flow in microvasculature. Currently known methods of measuring IMR provide inaccurate and/or imprecise results.

### SUMMARY

According to one aspect of the disclosed technology, there is provided a method executable by a system comprising a processor in communication with an intravascular pressure measurement device and with an extraluminal imaging device. The method comprises: acquiring, by the extraluminal imaging device, a first set of contrast-agent angiographic images of a blood vessel having a contrast agent therein; generating a reconstructed geometry object of the blood vessel based on the first set of contrast-agent angiographic images; based on the first set of contrast-agent angiographic images, estimating a first blood flow in the blood vessel; acquiring, by the intravascular pressure measurement device, a first set of values of the blood pressure; determining, by the processor, a first set of output parameters based on the first set of values of the blood pressure and the first blood flow, by implementing a first physical model of blood distribution in the blood vessel using the reconstructed geometry object; and generating a reconciled hemodynamic parameter based on the first set of output parameters.

The first set of contrast-agent angiographic images and the first set of values of the blood pressure may be obtained during a hyperemic state of the blood vessel, the blood vessel having a hyperemic agent therein. In at least one embodiment, the first set of values of the blood pressure may be acquired as a function of location within the blood vessel. The first set of values of the blood pressure may be a function of a location within the blood vessel. The method may further comprise acquiring, by the extraluminal imaging device, a first set of pressure-measurement angiographic images of the blood vessel. The first set of values of the blood pressure may be registered with the first set of the pressure measurement angiographic images. The method may further comprise determining, by the processor, the first set of output parameters based on the first set of values of the blood pressure registered with the first set of the pressure-measurement angiographic images. The method may further comprise, when the blood vessel has a hyperemic agent therein: acquiring, by the extraluminal imaging device, a second set of contrast-agent angiographic images of a blood vessel having the contrast agent and hyperemic agent therein; based on the second set of contrast-agent angiographic images, estimating a second blood flow in the blood vessel; acquiring, by the intravascular pressure measurement device, a second set of values of the blood pressure within the blood vessel having the hyperemic agent therein; determining, by the processor, a second set of output parameters based on the second set of values of the blood pressure and the second blood flow, by implementing a second physical model of the blood distribution in the blood vessel and using the reconstructed geometry object; and wherein generating the reconciled hemodynamic parameter further comprises: adjusting the reconciled hemodynamic parameter based on the first set of the output parameters and the second set of the output parameters. The second set of values of the blood pressure may be a function of a location within the blood vessel. The method may further comprise acquiring, by the extraluminal imaging device, the second set of pressure-measurement angiographic images of the blood vessel and registering the second set of values of the blood pressure with the second set of the pressure-measurement angiographic images. The method may further comprise determining the second set of output parameters, by the processor, based on the second set of values of the blood pressure registered with the second set of the pressure-measurement angiographic images.

The method may further comprise, when the blood vessel has a hyperemic agent therein: acquiring, by the intravascular pressure measurement device, a second set of values of the blood pressure as a function of a location within the blood vessel having the hyperemic agent therein, and acquiring, by the extraluminal imaging device, a second set of pressure-measurement angiographic images of the blood vessel; acquiring, by the extraluminal imaging device, a second set of contrast-agent angiographic images of the blood vessel having the contrast agent and the hyperemic agent therein; based on the second set of contrast-agent angiographic images, estimating a second blood flow in the blood vessel; determining, by the processor, a second set of output parameters based on the second set of values of the blood pressure, which may be registered with the second set of the pressure-measurement angiographic images, by implementing a second physical model of the blood distribution in the blood vessel and using the reconstructed geometry object; and wherein generating the reconciled hemodynamic parameter further comprises: adjusting the reconciled hemodynamic parameter based on the first set of the output parameters and the second set of the output parameters.

The method may further comprise, after a stent has been installed in the blood vessel or after another percutaneous coronary intervention: acquiring, by the extraluminal imaging device, a third set of contrast-agent angiographic images of the blood vessel having a stent and the contrast agent therein; based on the third set of contrast-agent angiographic images, estimating a third blood flow in the blood vessel; acquiring, by the intravascular pressure measurement device, a third set of values of the blood pressure within the blood vessel; determining, by the processor, a third set of output parameters based on the third set of values of the blood pressure and the third blood flow, by implementing a third physical model of the blood distribution in the blood vessel using the reconstructed geometry object; and wherein the adjusting the reconciled hemodynamic parameter is further based on the third set of the output parameters. The third set of values of the blood pressure may be a function of and/or measured as function of a location within the blood vessel. The method may further comprise acquiring, by the extraluminal imaging device, the third set of pressure-measurement angiographic images of the blood vessel and registering the third set of values of the blood pressure with the third set of the pressure-measurement angiographic images. The method may further comprise determining the third set of output parameters, by the processor, based on the third set of values of the blood pressure registered with the third set of the pressure-measurement angiographic images. The third set of contrast-agent angiographic images, the third set of values of the blood pressure and the third set of pressure-measurement angiographic images may be obtained during a hyperemic state of the blood vessel, the blood vessel having the hyperemic agent therein. At least a third period of time may elapse after acquiring of the third set of the contrast-agent angiographic images before acquiring the third set of pressure-measurement angiographic images and the third set of values of the blood pressure.

The method may further comprise: acquiring, by the extraluminal imaging device, a fourth set of contrast-agent angiographic images of the blood vessel having the stent and the contrast agent therein; based on the fourth set of contrast-agent angiographic images, estimating a fourth blood flow in the blood vessel; acquiring, by the intravascular pressure measurement device, a fourth set of values of the blood pressure within the blood vessel; determining, by the processor, a fourth set of output parameters based on the fourth set of values of the blood pressure and the fourth blood flow, by implementing a fourth physical model of the blood distribution in the blood vessel using the reconstructed geometry object; and wherein the adjusting the reconciled hemodynamic parameter is further based on the fourth set of the output parameters. The fourth set of values of the blood pressure may be a function of and/measured as a function of a location within the blood vessel. The method may further comprise acquiring, by the extraluminal imaging device, the fourth set of pressure-measurement angiographic images of the blood vessel and registering the fourth set of values of the blood pressure with the fourth set of the pressure-measurement angiographic images. The method may further comprise determining the fourth set of output parameters, by the processor, based on the fourth set of values of the blood pressure registered with the fourth set of the pressure-measurement angiographic images.

In some embodiments, the acquiring of the fourth set of values of the blood pressure and the fourth set of pressure-measurement angiographic images is executed after at least a fourth period of time elapsed since acquiring of the fourth set of the contrast-agent angiographic images.

The acquiring of the first set of values of the blood pressure may be simultaneous with the acquiring of the first set of pressure-measurement angiographic images of the blood vessel. In at least one embodiment, the acquiring of the second set of values of the blood pressure may be simultaneous with the acquiring of the second set of pressure-measurement angiographic images of the blood vessel. The acquiring of the third set of values of the blood pressure may be simultaneous with the acquiring of the third set of pressure-measurement angiographic images of the blood vessel. In at least one embodiment, the acquiring of the fourth set of values of the blood pressure may be simultaneous with the acquiring of the fourth set of pressure-measurement angiographic images of the blood vessel.

The method may further comprise: determining, by the processor, a second set of output parameters based on a second set of values of the blood pressure having a hyperemic agent therein and without the contrast agent (in other terms, acquired after a second period of time elapsed since acquiring the first set of contrast-agent angiographic images), and a second blood flow determined from a second set of contrast-agent angiographic images acquired when the contrast agent and a hyperemic agent are in the blood vessel; and wherein generating the reconciled hemodynamic parameter further comprises adjusting the reconciled hemodynamic parameter based on the first set of the output parameters and the second set of the output parameters. The method may further comprise acquiring the second set of pressure-measurement angiographic images of the blood vessel and registering the second set of values of the blood pressure with the second set of pressure-measurement angiographic images of the blood vessel.

The method may further comprise, after a stent has been installed in the blood vessel, determining, by the processor, a third set of output parameters based on: a third set of values of the blood pressure acquired with acquiring of a third set of pressure-measurement angiographic images after at least a third period of time elapsed since acquiring the second set of contrast-agent angiographic images and a third blood flow determined from a third set of contrast-agent angiographic images acquired when the contrast agent is in the blood vessel; and wherein adjusting the reconciled hemodynamic parameter is further based on the third set of output parameters.

The method may further comprise determining, by the processor, a fourth set of output parameters based on a fourth blood flow determined from a fourth set of contrast-agent angiographic images acquired when the blood vessel has the contrast agent and the hyperemic agent therein, and a fourth set of values of the blood pressure, which may be registered with the acquired fourth set of pressure-measurement angiographic images, when the blood vessel has the hyperemic agent and does not have the contrast agent therein (after at least a fourth period of time elapsed since acquiring the fourth set of contrast-agent angiographic images); and wherein adjusting the reconciled hemodynamic parameter is further based on the fourth set of output parameters.

The first set of output parameters may comprise pressure estimates along a centerline of the blood vessel. The first physical model may be a three-dimensional physical model, a two-dimensional model, one-dimensional model, or a zero-dimensional model. The first physical model, second physical model, third physical model, and/or fourth physical model may be three-dimensional models, two-dimensional models, one-dimensional models, or zero-dimensional models. The first physical model, second physical model, third physical model, and/or fourth physical model may be hybrid physical model(s). The first physical model may be a machine-learning model. The second physical model, third physical model and/or fourth physical model may be machine-learning model(s).

The method may further comprise generating a reconciliated pressure field as a function of a location in the blood vessel. The method may further comprise generating and displaying a combined output image comprising reconciliated pressure values determined based on the first output parameters and superimposed with the reconstructed geometry image and representing a reconciliated pressure field. The method may further comprise generating and displaying a combined output image comprising visual representation of reconciliated pressure values superimposed with a reconstructed geometry object and representing a reconciliated pressure field. Estimating the first blood flow in the blood vessel may be further based on the reconstructed geometry object of the blood vessel. Adjusting of the reconciled hemodynamic parameter may be further based on the first blood flow. Adjusting of the reconciled hemodynamic parameter may be further based on at least one of the first blood flow, the second blood flow, the third blood flow and the fourth blood flow. In at least one embodiment, the first set of values of the blood pressure comprises values of proximal blood pressure and distal blood pressure. The method may further comprise displaying the reconciled hemodynamic parameter on a display.

The reconciliated pressure field may be generated as a function of a location in the blood vessel based on the first set of output parameters, based on the second set of output parameters, based on the third set of output parameters, and/or based on the fourth set of output parameters. The method may further comprise generating a reconciliated pressure field as a function of a location in the blood vessel based on the fourth set of output parameters. The method may further comprise generating a reconciliated pressure field as a function of a location in the blood vessel based on at least one of the first set of output parameters, the second set of output parameters, the third set of output parameters, and the fourth set of output parameters.

The reconciled hemodynamic parameter may be at least one of an index of microvascular resistance, a fractional flow reserve, a coronary flow reserve, a diastolic pressure ratio, an absolute flow, an absolute resistance, and a ratio of absolute resistance.

According to another aspect of the disclosed technology, there is provided a system comprising: an extraluminal imaging device configured to generate the first set of contrast-agent angiographic images of a blood vessel having a contrast agent therein; an intravascular pressure measurement device configured to measure values of the blood pressure, which may be measured as a function of location, within the blood vessel and generate endoluminal pressure data; and a processor configured to: generate a reconstructed geometry object of the blood vessel based on the first set of contrast-agent angiographic images; based on the first set of contrast-agent angiographic images, estimate a first blood flow in the blood vessel; determine a first set of output parameters based on the first blood flow and the first set of values of the blood pressure, which may be registered with a first set of the pressure-measurement angiographic images, by implementing a first physical model of blood distribution in the blood vessel using the reconstructed geometry object; and generate a reconciled hemodynamic parameter based on the first set of output parameters.

In at least one embodiment, the system further comprises a display configured to display the reconciled hemodynamic parameter and an image representing a reconciliated pressure field. The processor may be further configured to: based on a second set of contrast-agent angiographic images acquired when the blood vessel had the contrast agent and hyperemic agent, estimate a second blood flow in the blood vessel; determine a second set of output parameters based on a second set of values of the blood pressure acquired by the intravascular pressure measurement device, which may be registered with the second set of the pressure-measurement angiographic images acquired by the extraluminal imaging device, by implementing a second physical model of the blood distribution in the blood vessel and using the reconstructed geometry object; and adjust the reconciled hemodynamic parameter based on the first set of the output parameters and the second set of the output parameters.

The processor may be further configured to: based on a third set of contrast-agent angiographic images acquired when the blood vessel had the contrast agent, estimate a third blood flow in the blood vessel; determine a third set of output parameters based on a third set of values of the blood pressure acquired by the intravascular pressure measurement device, which may be registered with the third set of the pressure-measurement angiographic images acquired by the extraluminal imaging device, by implementing a third physical model of the blood distribution in the blood vessel and using the reconstructed geometry object; and adjust the reconciled hemodynamic parameter further based on the third set of the output parameters.

The processor may be further configured to: based on a fourth set of contrast-agent angiographic images acquired when the blood vessel had the contrast agent, estimate a fourth blood flow in the blood vessel; determine a fourth set of output parameters based on a fourth set of values of the blood pressure acquired by the intravascular pressure measurement device, which may be registered with the fourth set of the pressure-measurement angiographic images acquired by the extraluminal imaging device, by implementing a fourth physical model of the blood distribution in the blood vessel and using the reconstructed geometry object; and adjust the reconciled hemodynamic parameter further based on the fourth set of the output parameters.

According to a further aspect of the disclosed technology, there is provided a processor in communication with an extraluminal imaging device and an endoluminal data-acquisition device, the extraluminal imaging device is configured to acquire at least one angiographic image of a blood vessel, and the endoluminal data-acquisition device is configured to obtain values of blood pressure within the blood vessel, and wherein the processor is configured to: receive, from the extraluminal imaging device, a first set of contrast angiographic images of the blood vessel having a contrast agent; receive, from the endoluminal data acquisition device, a first set of values of the blood pressure, which may be a function of a location within the blood vessel, and, from the extraluminal imaging device, a first set of pressure-measurement angiographic images of the blood vessel without the contrast agent; generate a reconstructed geometry object of the blood vessel and a first blood flow based on the contrast-agent angiographic images; determine a first set of output parameters based on the first set of values of the blood pressure, which may be registered with the first set of the pressure-measurement angiographic images and based on the first blood flow, by implementing a first physical model of blood distribution in the blood vessel using the reconstructed geometry object; and generate a reconciled hemodynamic parameter based on the first set of output parameters.

The processor may be further configured to: determine a second set of output parameters based on a second set of contrast-agent angiographic images, a second set of values of blood pressure, which may be registered with a second set of pressure-measurement angiographic images, acquired when the blood vessel had a hyperemic agent; and wherein generating the reconciled hemodynamic parameter by the processor is further based on the second set of the output parameters. The processor may be further configured to: determine a third set of output parameters based on a third set of contrast-agent angiographic images, a third set of values of blood pressure, which may be registered with a third set of pressure-measurement angiographic images, acquired when the blood vessel had a stent therein; and wherein generating the reconciled hemodynamic parameter by the processor is further based on the third set of the output parameters.

The processor may be further configured to: determine a fourth set of output parameters based on a third set of contrast-agent angiographic images, a fourth set of values of blood pressure, which may be registered with a fourth set of pressure-measurement angiographic images, acquired when the blood vessel had a stent and a hyperemic agent therein; and wherein generating the reconciled hemodynamic parameter by the processor is further based on the fourth set of the output parameters.

According to a further aspect of the disclosed technology, there is provided a method executable by a system having a processor in communication with an extraluminal imaging device and an intravascular pressure measurement device, the method comprising: acquiring contrast-agent angiographic images when the blood vessel has a contrast agent therein; separately and for the same blood vessel and without contrast agent (or with a significantly lower concentration of the contrast agent), acquiring pressure-measurement angiographic images, acquiring intravascular pressure values (which may be measured by measuring proximal blood pressure and distal blood pressure, and may be measured as a function of location); reconstructing geometry of the blood vessel, and applying a physical model to a reconstructed geometry object, which may be registered with the intravascular pressure values; and generating a reconciled hemodynamic parameter, and adjusting the reconciled hemodynamic parameter based on the contrast-agent angiographic images, pressure-measurement angiographic images, and intravascular pressure values acquired when the blood vessel is in at least two of blood vessel states selected from: rest prior to percutaneous coronary intervention (PCI), hyperemia prior to PCI, hyperemia post-PCI, and rest post-PCI.

According to an aspect of the disclosed technology, the method as described herein comprises acquiring contrast-agent angiographic images in a blood vessel having a contrast agent, acquiring pressure-measurement angiographic images while measuring values of blood pressure; based on the contrast-agent angiographic images, generating a reconstructed geometry object of the blood vessel and estimating a blood flow in the blood vessel; determining output parameters based on the values of the blood pressure, which may be registered with the pressure-measurement angiographic images, and the blood flow, by implementing a physical model of blood distribution using the reconstructed geometry object; and generating a reconciled hemodynamic parameter based on the output parameters. The reconciled hemodynamic parameter is adjusted based on measurements performed at various blood vessel states.

### BRIEF DESCRIPTION OF THE DRAWINGS

Further features and advantages of the present disclosure will become apparent from the following detailed description, taken in combination with the appended drawings, in which:

FIG. 1A illustrates a portion of a blood vessel, such as a coronary artery with a guidewire, in accordance with at least one embodiment of the present disclosure;

FIG. 1B illustrates a portion of a coronary tree which comprises two coronary arteries;

FIG. 1C illustrates the coronary artery of FIG. 1A with stenoses;

FIG. 1D illustrates the coronary artery of FIG. 1A with a stent;

FIG. 2 schematically illustrates a system for determining and indication of a patient's condition, in accordance with at least one embodiment of the present disclosure;

FIG. 3 schematically illustrates an endoluminal data-acquisition device that may be used in the system of FIG. 2, in accordance with at least one embodiment of the present disclosure;

FIG. 4A illustrates a contrast-agent angiographic image acquired in accordance with at least one embodiment of the present disclosure;

FIG. 4B illustrates a mask of the angiographic image of FIG. 4A generated at a segmentation step, in accordance with at least one embodiment of the present disclosure;

FIG. 5A schematically illustrates a method for determining an indication of a patient's condition, in accordance with at least one embodiment of the present disclosure;

FIG. 5B illustrates other steps of the method of FIG. 5A, in accordance with at least one embodiment of the present disclosure;

FIG. 6A schematically illustrates a time diagram of implementation of a method for determining hemodynamic parameters, in accordance with at least one embodiment of the present disclosure;

FIG. 6B schematically illustrates a time diagram of another implementation of a method for determining hemodynamic parameters, in accordance with at least one embodiment of the present disclosure; and

FIG. 7 is a flowchart of a method for determining hemodynamic parameters, in accordance with at least one embodiment of the present disclosure.

It will be noted that throughout the appended drawings, like features are identified by like reference numerals.

### DETAILED DESCRIPTION

Various aspects of the present disclosure generally address one or more of the problems of determining hemodynamic parameters. The method and the system described herein is configured to generate reconciled hemodynamic parameters and images providing an indication of a patient condition, which are obtained based on blood flow computations, invasive pressure measurements and angiographic imagery. The method as described herein use intravascular pressure measurements, angiographic images and a physical model to generate reconciled hemodynamic parameters and a reconciled pressure field in the blood vessel.

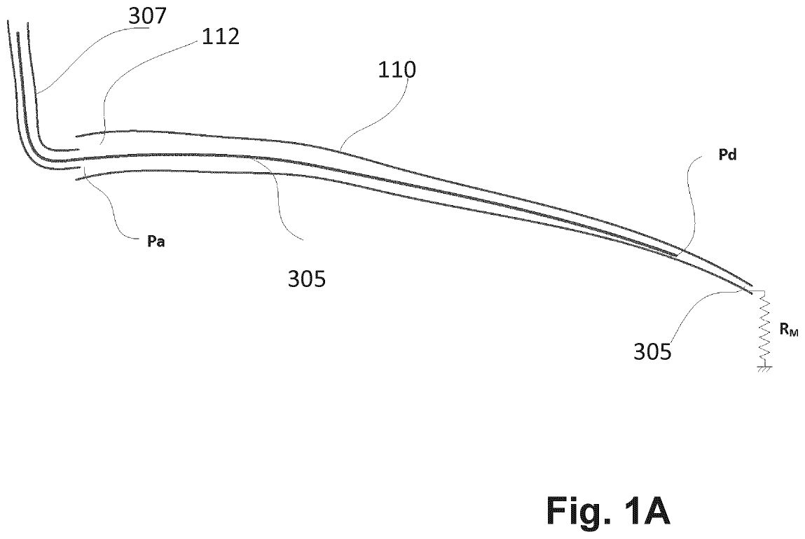

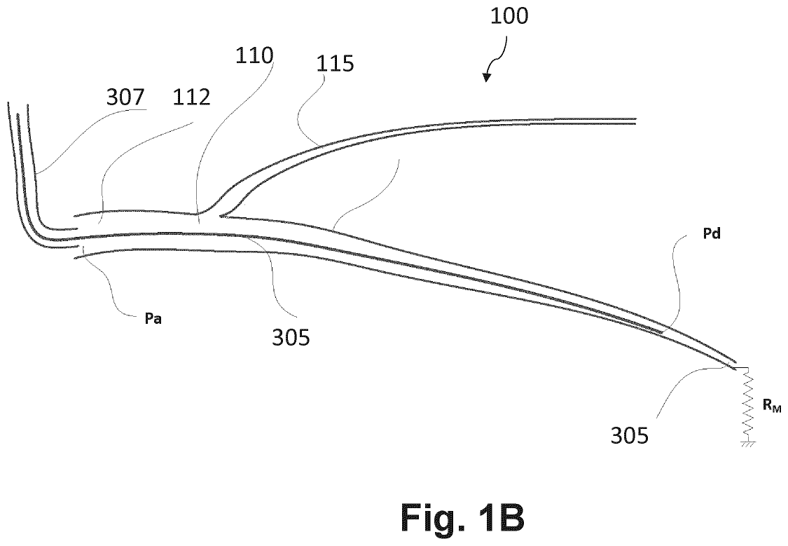

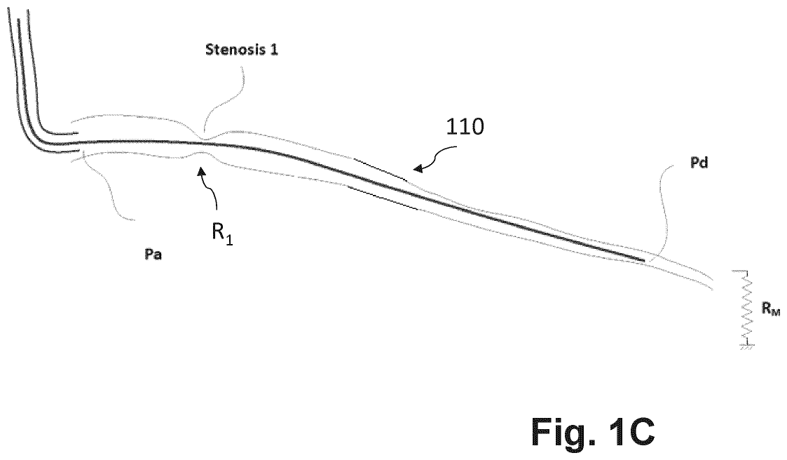

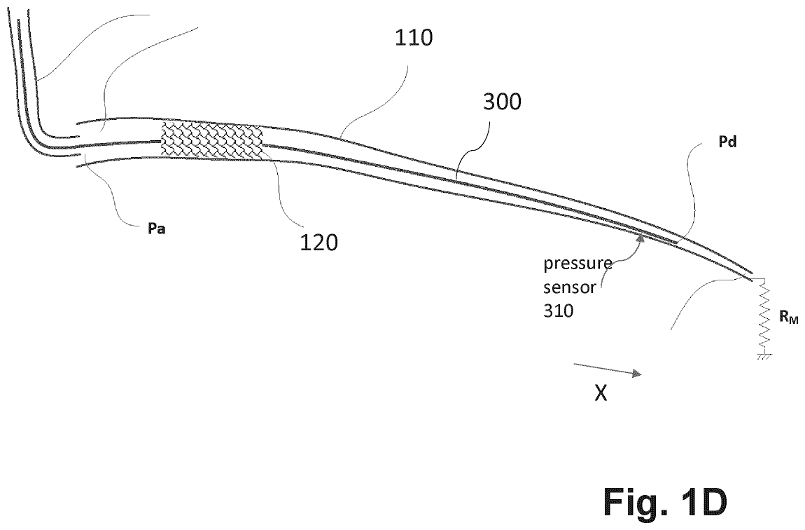

Referring now to the drawings, FIGS. 1A-1B illustrate portions of a blood vessel 110, and in particular, portions of a coronary artery having a lumen 112. FIG. 1B illustrates a portion of a coronary tree which comprises more than one blood vessel. FIG. 1C illustrates portions of the coronary artery having areas with stenosis. FIG. 1D illustrates the coronary artery of FIG. 1A with a stent 120. In FIGS. 1A-1D, a microvascular resistance RM is illustrated. An index of microvascular resistance (IMR) characterises the microvascular resistance of the blood vessel 110 and is related to the microvascular resistance RM as known in the art. In a simplified form, assuming coronary flow and myocardial flow are equal, and that the contribution of collateral flow is negligible, then IMR may be estimated as a distal coronary pressure divided by coronary flow mean transit time.

Currently known methods of determining the IMR are either invasive or non-invasive. The IMR may be determined invasively by measuring temperature and pressure in the coronary artery, while a liquid, which is colder than the patient's body temperature, is injected in the blood vessel 110. Alternative non-invasive methods may include using angiography to assess the coronary microvascular dysfunction and to calculate the IMR theoretically. Such methods, however, provide unreliable results due to many hypotheses or inaccurate assumptions need to be used. For example, to calculate the IMR based solely on the angiographic images, one needs to assume that the effect of the hyperemic agent on the microvascular resistance is equal to a population mean, or that the distal pressure may be accurately estimated by a value of fractional flow reserve (FFR) obtained non-invasively.

The method and the system described herein use both invasive and non-invasive steps, which permit building a model of the blood flow in a patient's blood vessel 110 and adjusting that model based on measurements performed within the blood vessel 110. In some embodiments, such adjustment of the model may be executed in real time based on real-time measurements performed within the blood vessel 110. The method and the system as described herein may help to improve the accuracy of obtained values of IMR.

It should be noted that coronary artery is an example of the blood vessel and the method as described herein may be used for any other blood vessel. For example, the method as described herein may be used to assess microvascular diseases of blood vessels located in other parts of the body, such as, for example, legs. Pulmonary circulation may be also evaluated using the method and system as described herein. Thus, when referred to herein, the “blood vessel” may be, for example, and without limitation, coronary artery, peripheral arteries, and vessels of the pulmonary circulation.

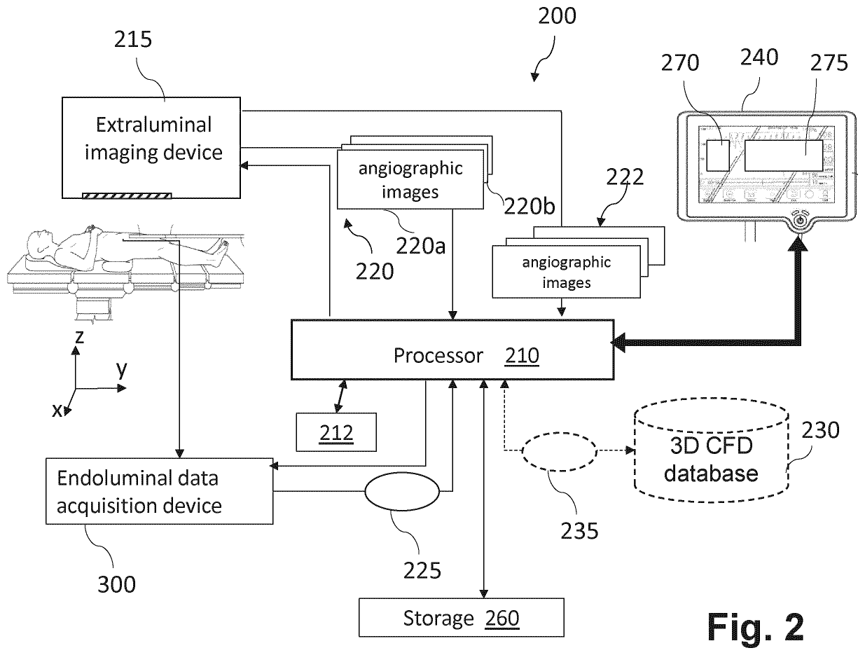

FIG. 2 schematically illustrates a system 200, in accordance with at least one embodiment of the present disclosure. The system 200 comprises a processor 210 which is in communication with an extraluminal imaging device 215. The extraluminal imaging device 215 is a non-invasive instrument and is configured to acquire one or more two-dimensional angiographic images 220, 222 (such as x-ray images) of a blood vessel 110. In at least one embodiment, the extraluminal imaging device 215 may be configured to acquire videos which have sequences of angiographic images. In some embodiments, the coordinate system xyz may be non-standard.

The system 200 also comprises an endoluminal data-acquisition device 300 (also referred to herein as an “intravascular pressure measurement device 300”) which is configured to be moved through the blood vessel 110 to obtain endoluminal pressure measurements 225 of the blood vessel 110 (in other words, values of blood pressure 225 in various locations of the blood vessel 110). The endoluminal data acquisition device 300 is connected to the processor 210, and transmits the pressure measurements 225 to the processor 210. The processor 210, based on the contrast-agent angiographic images 220 and the pressure measurements received from the endoluminal data-acquisition device 300, determines one or more reconciled hemodynamic parameter(s) and displays the reconciled hemodynamic parameter(s) on a display 240. The display 240 may be a touchscreen display and/or the system 200 may have an additional input device. Still referring to FIG. 2, the system 200 also has a memory 212 providing a place for storage of computer-executable instructions and is configured to store the computer-executable instructions executable by the processor 210. The memory 212 may be implemented as a computer-readable storage medium such as, for example, read-only memory, hard disk drives (HDDs), solid-state drives (SSDs), and flash-memory cards. A database 230 may be optionally used as described below. The processor 210 also has ports of communication for performing logical operations on signals.

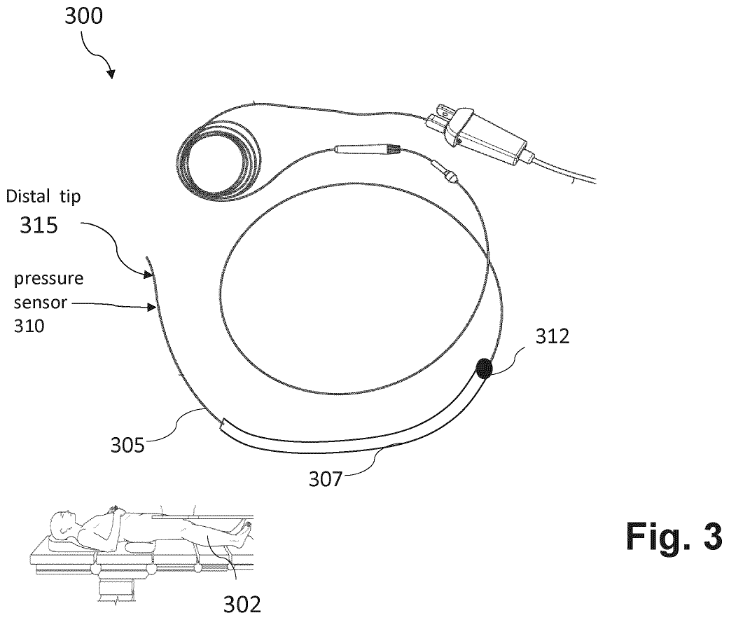

FIG. 3 illustrates a non-limiting example of the endoluminal data-acquisition device 300 that may be used in the system 200 to acquire pressure measurement, so-called pressure guidewire. The endoluminal data-acquisition device 300 comprises a distal pressure sensor 310 which is located at a distal end 315 on a pressure guidewire 305 or a catheter and is moved through a cardiovascular system of a patient 302 to reach the blood vessel 110. Referring also to FIGS. 1A-1C, the endoluminal data-acquisition device 300 measures pressure at different locations within the blood vessel 110. The endoluminal data-acquisition device 300 acquires a position of the distal pressure sensor 310 with regards to the blood vessel's geometry including, but not limited to, the blood vessel's length. The distal pressure sensor 310 of the endoluminal data-acquisition device 300 is thus configured to measure the values of the blood pressure 225 as a function of a location within the blood vessel 110. The location may be measured as a coordinate (x, y) and/or a longitudinal position (l) along the blood vessel 110. In some embodiments, the values of the blood pressure 225 are measured as a function of a distance (for example, within the vessel 110) from a reference point in the vessel 110, such as, for example, a point of entry of the distal pressure sensor 310 into the vessel 110. In at least one embodiment, the values of the blood pressure 225 are measured as a function of time (t) and are timestamped.

Catheter 307 has a proximal pressure sensor 312 that may be located, during the measurements of the distal pressure inside the blood vessel 110, at an entrance to the blood vessel 110. The measurements of the distal pressure 527 and proximal pressure 529 (referred to collectively as the values of the blood pressure 225) and are performed when the distal pressure sensor 310 and the proximal pressure sensor 312 are located at approximately the same height.

Thus, the catheter 307 with the guidewire 305 located inside the catheter 306 together measure the proximal pressure Pₐ and the distal pressure P_{d}. The proximal pressure and the distal pressure may be measured simultaneously (synchronously). In at least one embodiment, the signals related to and representing the values of the distal pressure 527 and proximal pressure 529 are measured synchronously and sampled at a pre-determined frequency. In other terms, the two signals of the two measurements of the distal pressure 527 and proximal pressure 529 are synchronized and sampled at the pre-determined frequency. For example, and without limitation, such pre-determined frequency may be between 50 and 1100 Hz.

In at least one embodiment, the endoluminal data-acquisition device 300 may be introduced into the blood vessel 110 via a proximal end in the blood vessel 110 until the distal end 315 reaches a distal point of the blood vessel 110 under investigation, and then the endoluminal data-acquisition device 300 is pulled back towards the proximal end of the blood vessel 110. The endoluminal pressure data 225 may be obtained during such pull back of the endoluminal data-acquisition device 300. As illustrated in FIG. 2, the endoluminal data-acquisition device 300 is connected to the processor 210 which receives, in the in-vivo regime, sequentially, the endoluminal pressure data 225 from the endoluminal data-acquisition device 300.

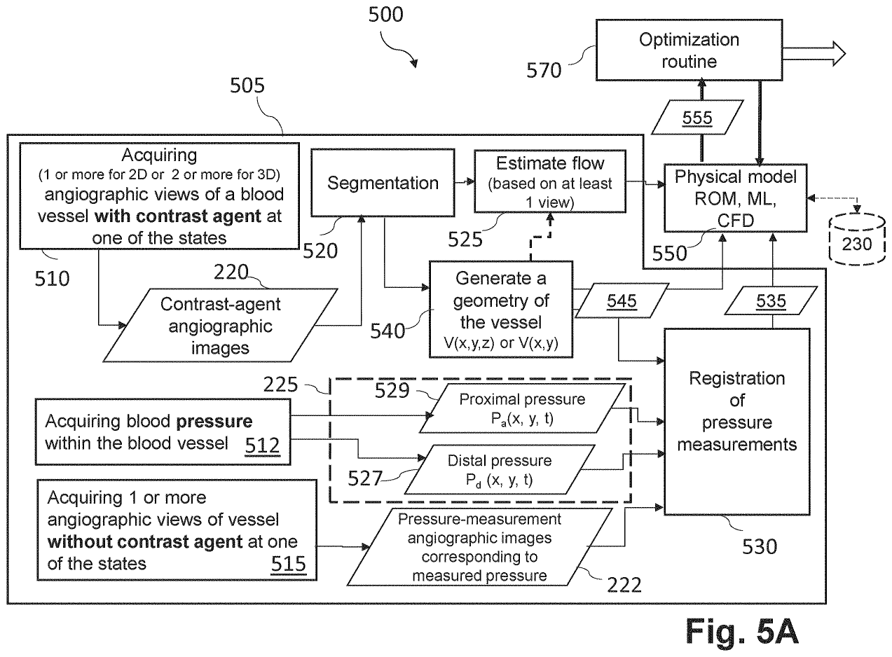

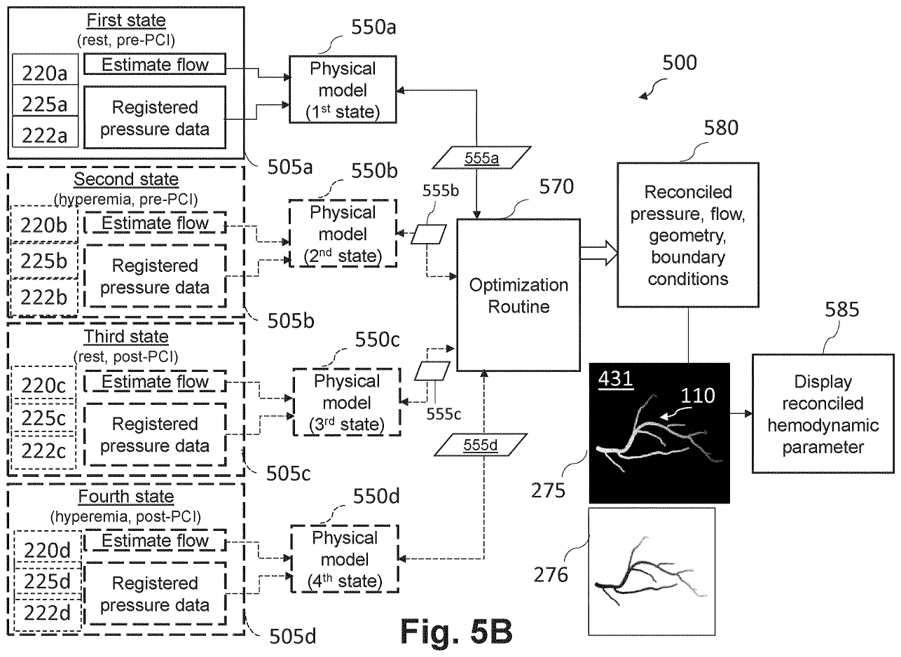

FIGS. 5A and 5B schematically illustrate steps of a method 500 for determining hemodynamic parameters, in accordance with at least one embodiment of the present disclosure. In addition to obtaining values of the blood pressure 225 with the endoluminal data-acquisition device 300, the method 500 comprises acquiring, by the extraluminal imaging device 215, and use of angiographic images 220, 222 obtained in two conditions: with and without contrast agent in the blood vessel 110. Contrast-agent angiographic images 220 are acquired when the blood vessel 110 has a contrast agent therein. Pressure-measurement angiographic images 222 (which may be also referred to as “guidewire-position angiographic images”) are acquired without the contrast agent in the blood vessel 110, while the blood pressure 225 is measured with the endoluminal data-acquisition device 300.

When referred to herein, the contrast-agent angiographic images 220 obtained/acquired “with the contrast agent” means that contrast-agent angiographic images 220 are acquired while or just before the contrast agent is introduced into the blood vessel 110. When the blood vessel 110 has the contrast agent, the contrast agent is visible on the contrast-agent angiographic images 220 to the user (practician) and/or the blood vessel with the contrast agent can be segmented and separated (distinguished) from the background by the processor 210.

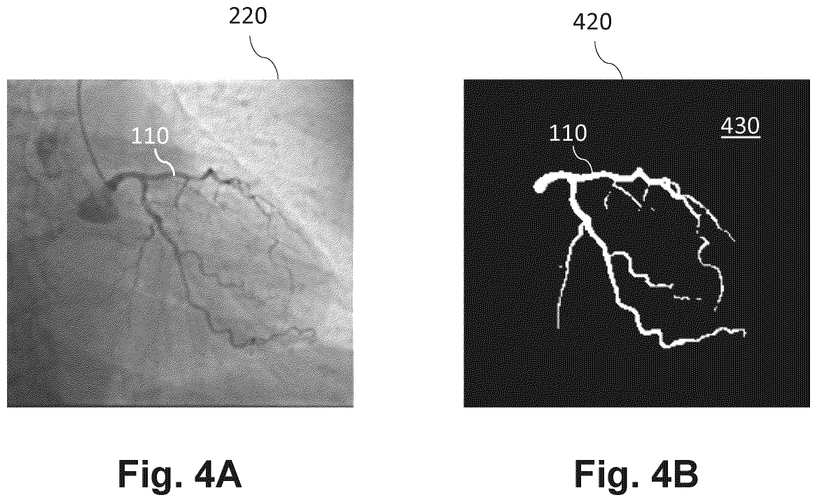

FIG. 4A illustrates a contrast-agent angiographic image 220 taken in accordance with at least one embodiment of the present disclosure. The contrast agent helps to discern the blood vessel 110 from the background in the angiographic image and shows the blood vessel 110 “with a contrast”. The contrast agent may be added, for example, by injecting the contrast agent directly into the blood vessel 110 or by injecting a bolus of the contrast agent through a diagnostic catheter at the ostium of the coronary sinus. The contrast agent may be, for example, an iodinated contrast containing iodine.

The contrast agent may be added into the blood vessel 110 (lumen of the blood vessel) to obtain a set of contrast-agent angiographic images 220. The contrast-agent angiographic images 220 may be acquired when a concentration of the contrast agent (also referred to herein as a first concentration of the contrast agent”) in the blood vessel is high enough to allow the blood vessel to be distinguishable in the contrast-agent angiographic images 220.

In at least one embodiment, the extraluminal imaging device 215 captures the contrast-agent angiographic images 220 when the contrast agent flows in the blood vessel 110 during a contrast-agent period of time. The contrast-agent period of time (which may be counted since injecting the contrast agent or the bolus of the contrast agent) is long enough for obtaining (and recording) images with the contrast agent filling and propagating through the coronary tree before all the contrast agent diffuses. When the contrast agent is flowing in the blood vessel 110, concentration of the contrast agent in the blood vessel 110 is sufficient enough to provide a distinguishable contrast between the blood vessel's image and the surroundings of the blood vessel 110 image in the acquired contrast-agent angiographic image 220. The contrast-agent period of time may be, for example, 3 to 5 seconds, or, for example, less than 10 seconds. The contrast-agent period of time may be predetermined, or, alternatively, an operator (medical practician) may stop introducing the contrast agent into the blood vessel 110 when the contrast-agent angiographic image(s) 220 displayed on the display 240 has enough contrast (in other terms, has reached a level of contrast allowing to clearly discern the blood vessel 110 from its environment).

The pressure-measurement angiographic images 222 are obtained/acquired “without the contrast agent” meaning that the pressure-measurement angiographic images 222 are acquired when the blood vessel 110 does not have any contrast agent in the blood vessel 110 due to its physical absence or because the contrast-agent period of time has lapsed since the contrast agent has been introduced into the blood vessel 110 and the concentration of the contrast agent has faded.

The pressure-measurement angiographic images 222 are acquired when the concentration of the contrast agent in the blood vessel 110 has decreased and reached a second concentration of the contrast agent, which may happen after the contrast-agent period of time and as fast as several seconds after the injection of the contrast agent or the injection of the bolus with the contrast agent into the blood vessel 110. In other words, pressure-measurement angiographic images 222 are obtained when the concentration of the contrast agent in the blood vessel is the second concentration of the contrast agent which is significantly lower than the first concentration of the contrast agent, e.g. zero or near zero. Because the injection of contrast agent in the catheter disrupts the aortic pressure signal (Pa), the pressure measurement must be taken either before the injection of the contrast agent, or a sufficient amount of time after the injection of the contrast agent, when the aortic pressure measurement is restored. In the case where the pressure measurement is taken before the contrast agent injection, the contrast agent may be used to trigger the saving of retrospective pressure signals, for example by saving the mean Pa and Pd of the last 1 to 10 heartbeats recorded.

Referring again to FIG. 2, extraluminal measurements (contrast-agent angiographic images 220 and pressure-measurement angiographic images 222) and intraluminal pressure measurements 527, 529 may be acquired at one, two, three or four of four states of the patient's blood vessel 110. These states of the blood vessel 110 depend on whether the blood vessel 110 is at rest or hyperemia, and whether the blood vessel 110 has already had a percutaneous coronary intervention.

To induce a hyperemia, a hyperemic agent (which may be also referred to as a “hyperemia-inducing agent”) may be introduced into the blood vessel 110. While the contrast agent may induce hyperemia by itself, preferably, a hyperemic agent is introduced into the blood vessel 110 to induce a hyperemic state of the blood vessel. Nevertheless, in some embodiments, the hyperemic agent may be the contrast agent. The hyperemic agent may be introduced, for example, by constant intravenous infusion of adenosine. It is also possible to induce transient hyperemia with intra-coronary (IC) bolus of adenosine or other drugs.

When the contrast-agent angiographic images 220 need to be taken during the hyperemic state of the blood vessel 110, the user (operator) would have to synchronize the injection of the hyperemic drug with the injection of the contrast agent, as the duration of IC-induced hyperemia is quite short. Alternatively, the contrast agent may be introduced using the contrast agent bolus, while the hyperemic agent may be introduced by constant intravenous infusion of adenosine.

In the first state of the blood vessel 110, the measurements may be obtained when the blood vessel 110 is at rest (in other terms, “in a rest state”). In the rest state, the blood vessel 110 has no stress induced by the hyperemic agent, and the measurements are performed without introduction (application) of the hyperemic agent.

In the second state, the blood vessel 110 is under stress condition, i.e., under full hyperemic condition. As described above, the hyperemic state of the blood vessel 110 (referred to also as “hyperemic condition”) may be induced by the hyperemic agent. The measurements such as angiographic images 220, 222 and intravascular pressure measurements 225 may be taken when the blood vessel 110 has the hyperemic agent therein following (and due to) the introduction of the hyperemic agent into the blood vessel 110. In other words, in the second state, the blood vessel 110 may have a concentration of a hyperemic agent in the blood vessel which induces the stress in the blood vessel 110.

The concentration and the amount of the hyperemic agent in the blood vessel 110 reduces with time, and therefore the effect of the hyperemic agent reduces with time. After a period of time, which may be, for example, several seconds, and without additional introduction of the hyperemic agent, the blood vessel 110 is again in the rest state. The concentration of the hyperemic agent in the blood in the second state (hyperemic state) is higher than the concentration of the hyperemic agent in the first state (rest state), and in the preferred embodiments the concentration of the hyperemic agent in the blood vessel 110 in the rest state is zero or close to zero, because no hyperemic agent has been recently induced or the effect of the hyperemic agent has been faded.

As it is known in the art, and illustrated in FIG. 1D, a stent 120 may be installed to open blood vessels 110 in the heart that have been narrowed by plaque buildup due to a condition known as atherosclerosis. Such an intervention may be referred to as a percutaneous coronary intervention (PCI). As an alternative to installation of the stent 120, angioplasty may be used as the PCI. In angioplasty, a balloon may be inflated for a short time to push the plaque back against the wall of the coronary artery to improve the blood flow. For example, drug coated balloons (DSG) may be used during the PCI.

In method 500 described herein, the first and second states of the blood vessel 110 may be induced prior to any PCI. The first state may be therefore also referred to herein as a “pre-PCI rest state” and the second state may be also referred to herein as a “pre-PCI hyperemic state”.

The third and the fourth states of the blood vessel 110 may be implemented after the PCI. In the third state, the blood vessel 110 is at rest and after the PCI. The third state may be also referred to herein as a “post-PCI rest state”, where the measurements are obtained when the blood vessel 110 is without stress (i.e. without hyperemia conditions) and after the structure such as a stent has been installed. In the third state, the angiographic images are taken, and blood pressure is measured after the stent 120 (or another structure related to PCI) has been installed. In the fourth state, referred to herein as a “post-PCI hyperemic state”, the blood vessel 110 is under stress (i.e., under full hyperemic condition) and after the PCI.

Now referring to FIGS. 5A and 5B, the method 500 and the preliminary routine 505 of the method 500 may be implemented for one or more states of the blood vessel 110. In the context of the present specification, the term “routine” refers to a subset of the computer-executable program instructions of the method 500 that is executable by the processor 210 to perform the functions explained below in association with the various routines.

In at least one embodiment, each preliminary routine 505a, 505b (also referred to as the “preliminary routine 505”) is executed for one state of the blood vessel 110 and comprises acquisition of data at steps 510, 512, 515 (also referred to herein as “acquisition data steps”), for subsequent transmission to the processor 210. For example, for each additional state, one or more acquisition data steps may be executed. In some embodiments, in a subsequent state, executed after the first state, only two measurement steps may be executed, such as, for example: step 510 (acquiring angiographic views of the blood vessel with contrast agent) and step 512 (acquiring blood pressure within the blood vessel), or step 512 and step 515 (acquiring angiographic views of vessel without contrast agent). Still at the preliminary routine, the flow is estimated at step 525, the pressure and geometry data 535 is generated at step 540, and the pressure and geometry data 535 is generated at step 530.

At the contrast-agent image acquisition step 510, one or more sets of contrast-agent angiographic images 220 (such as x-ray images) of a blood vessel 110 are obtained while the blood vessel 110 has the contrast agent. The contrast-agent angiographic images 220 are two-dimensional images, taken from at least two different angles (in other words, in different planes) with respect to the blood vessel 110. For example, a first contrast-agent angiographic image 220 may be taken in a geometric plane which is approximately parallel to the blood vessel 110, and a second contrast-agent angiographic image 220 may be taken in a second plane which may be, for example, perpendicular or, for example, at an angle between 60 and 130 degrees to the plane of the first contrast-agent angiographic image 220. The second plane may be also approximately parallel to the blood vessel 110. The images of the same blood vessel 110, taken in different planes may be also referred as “views”. Each view corresponds to images taken in one geometric plane. The contrast-agent angiographic images 220 may be saved in storage 260.

Each contrast-agent angiographic image 220a of the set of contrast-agent angiographic images 220 may be obtained from a video with angiographic images obtained with the extraluminal imagining device 215 and recorded when the contrast agent is in the blood vessel 110. Each video and therefore contrast-agent angiographic image 220a may correspond to one view (at a particular angle) of blood vessel 110.

As described above, the contrast agent, introduced into the blood vessels 110 during the angiographic imagery at the contrast-agent image acquisition step 510, helps distinguishing the blood vessels from the background. In other words, the contrast-agent angiographic images 220 taken with the contrast agent in the blood vessels permit to clearly distinguish the blood vessels, making the blood vessels 110 visible to the user when the contrast-agent angiographic images 220 are displayed and permitting to determine location and geometry of the blood vessels.

After at least a first period of time has elapsed since introduction of the contrast agent (and acquiring of the set of the contrast-agent angiographic images 220), and the blood vessel 110 is without the contrast agent, the pressure-measurement angiographic images 222 (such as, for example, x-ray images) are acquired (step 515) and a set of values of the blood pressure 225 are acquired by the intravascular pressure measurement device 300 in the blood vessel 110 (step 512). In at least one embodiment, the pressure-measurement angiographic images 222 and a set of values of the blood pressure 225 are acquired simultaneously (synchronously). In at least one preferable embodiment, the values of the blood pressure are acquired simultaneously with the acquiring of the second set of pressure-measurement angiographic images of the blood vessel having a hyperemic agent therein.

In other words, the endoluminal data-acquisition device 300 measures the proximal pressure Pₐ and distal pressure P_{d} (illustrated in FIG. 1C) as a function of a location (for example, a distance from an entry point of the endoluminal data-acquisition device 300 into the blood vessel 110) within the lumen of the blood vessel 110 (step 512 in FIG. 5A). In some embodiments, the measurements of the pressure—the proximal pressure Pₐ and the distal pressure P_{d}—may be measured as a function of time. In some embodiments, the location (or position) may be described as a coordinate and the proximal pressure and the distal pressure may be measured as a function of the coordinates. The acquired set of the values of the blood pressure 225 comprises values of distal blood pressure 527 and values of the proximal blood pressure 529 measured as a function of location within the blood vessel 110 and, in some embodiments, time. Still referring to FIG. 5A, simultaneously with the measurement of distal blood pressure 527 and values of the proximal blood pressure 529 within the blood vessel 110 using the endoluminal data-acquisition device 300, the system 200 acquires pressure-measurement angiographic images 222 of the same blood vessel 110 (step 515 in FIG. 5A). The measurements may be timestamped.

Measured at step 515 without the contrast agent, the pressure-measurement angiographic images 222 do not permit to clearly distinguish the blood vessels and the user (such as a clinician) cannot see the blood vessels 110 if the pressure-measurement angiographic image 222 is displayed. However, each pressure-measurement angiographic image 222 illustrates a tip of the endoluminal data-acquisition device 300 and its spatial location and therefore the user may see where (at which location within the blood vessel 110) the pressure has been measured by the endoluminal data-acquisition device 300.

The pressure-measurement angiographic images 222 are two-dimensional images, taken from one or more different angles with respect to the blood vessel 110, while the pressure measurements with the endoluminal data-acquisition device 300 are performed in the blood vessel 110. The pressure-measurement angiographic images 222 may correspond to one or more views and may be also obtained from a video recorded by the extraluminal imaging device 215. Correspondence of the pressure-measurement angiographic images 222 to the measurements of the blood pressure 225 (values of the distal pressure 527 and pressure measurements 529) may be provided, for example, by time stamps.

To determine a pressure field within the blood vessel 110, the method 500 and the system 200 as described herein merge (in other terms, superimpose or overlay) the data obtained during the pressure measurements 527, 529, 222 with a geometry data of the blood vessel 110, obtained from the contrast-agent angiographic images 220 at step 540.

The geometry of the blood vessel(s) 110 may become clearly detectable due to the segmentation step 520 where the segmentation of the contrast-agent angiographic images 220, acquired earlier at the contrast-agent image acquisition step 510 is performed by the processor 210 (FIGS. 2 and 5A). During the segmentation step 520, at least one contrast-agent angiographic image 220 per view is processed to generate a corresponding mask 420. The mask 420 identifies parts of the contrast-agent angiographic image 220 corresponding to background 430 and parts of the contrast-agent angiographic image 220 corresponding to the blood vessel(s) 110. FIG. 4B illustrates a mask of the angiographic image of FIG. 4A generated at the segmentation step 520, in accordance with at least one embodiment of the present disclosure. In FIG. 4B, the blood vessel 110 is the coronary artery. In at least one embodiment, the processor 210 may segment only one image per view (sequence) to perform a 3D geometry reconstruction of the blood vessel 110, and two images per view sequence to estimate the flow.

For example, the mask 420 may have the same size/shape as the contrast-agent angiographic images 220 with the background 430 shown with black color (which may be referred to as “0” or “(0, 0, 0)” in RGB) and the blood vessel(s) 110 such as, for example, arteries, shown with white color (which may correspond to “1” or “255”, depending on the number of bits). In FIG. 4B, the mask has the blood vessels shown with white color and the background 430 around the blood vessels 110 is black.

At step 540, geometric vessel coordinates (also referred to herein as a “geometry”) of the blood vessel 110 are reconstructed based on the output of the segmentation step 520 obtained from the contrast-agent angiographic images 220. In at least one embodiment, at step 540, a reconstruction routine generates, as an output, a reconstructed geometry object 545 which provides the geometric vessel coordinates of the blood vessel 110.

The geometric vessel coordinates may be three-dimensional (3D), and may be represented as V(x, y, z). In at least one embodiment, the 3D geometry of the blood vessel 110 may be reconstructed based on the contrast-agent angiographic images 220 of the blood vessel 110. Various methods of reconstructing the 3D geometry may be used. Some of the methods are described in publication Çimen, S., Gooya, A., Grass, M., & Frangi, A. F. (2016). Reconstruction of coronary arteries from X-ray angiography: A review. Medical Image Analysis, 32, 46-68. For example, there are model-based methods that may be, for example, forward projection, back projection, four-dimensional, multi-view or vascular lumen reconstruction. Tomographic methods, such as so-called “gated” and motion-compensated methods of 3D reconstruction may be used.

Depending on the number of the sets (views) of contrast-agent angiographic images 220 acquired, two-dimensional (2D) reconstructed geometry or three-dimensional (3D) reconstructed geometry of the blood vessel 110 may be generated, as described below. The 3D reconstruction may be based on two or more angiographic views 510 that have been segmented at step 520. In two dimensions, the system may use one or more angiographic views acquired at step 510, then segmented at step 520, and then the system generates a two-dimensional geometry of the blood vessel 110. For example, two or more sets of contrast-agent angiographic images 220 may help to generate 2D or 3D reconstructed geometry of the blood vessel 110.

In at least one embodiment, the initial 3D model of the blood vessel 110 may be obtained by determining a two-dimensional (2D) projection of the vessel's image and determining a 3D model using an elastic registration. The elastic registration provides localized stretching of images to correct local non-linear deformations. In at least one embodiment, the system and the method described herein may use a generative neural network such as a generative adversarial network (GAN).

The 3D parameters of the vessel may be also obtained using computer tomography angiography (CTA). The CTA is a type of medical test that combines a scan using a computer tomography with an injection of a dye to produce pictures of blood vessels and tissues in a part of the patient's body. The dye is injected through an intravenous (IV) line started in an arm or a hand. Other methods, such as an iterative model reconstruction, may be used to obtain the 3D model of the artery.

The 3D reconstruction may be used to estimate volume to calculate the flow. It may be also used as input data for a computational fluid dynamics (CFD) model described below. For example, the diameter along the blood vessels may be used to estimate resistances. The reconstructed 3D geometry data of the blood vessel, obtained from the sequences of the angiographic images, may also help to determine the blood flow (Q). By estimating the volume filled with contrast agent as a function of time, one may obtain volume change versus time change (dV/dt) which is, by definition, the blood flow (Q) measured in cubic meters divided by seconds (m³/s).

In at least one embodiment, at step 540, instead of 3D reconstruction of the geometric vessel coordinates of the blood vessel 110, lower dimensional reconstruction may be used with lower dimensionality embeddings. For example, a two-dimensional (2D) reconstruction may be performed at step 540 to obtain the reconstructed geometry object 545 by a 2D model such as, for example, and without limitation, 2D embedding or a model that is projected onto a plane. One-dimensional (1D) reconstruction may be performed by calculating values at nodes and along one line. Alternatively, a zero-dimensional model (0D) may be used where values are calculated at nodes.

In at least one embodiment, the contrast-agent angiographic images 220 received by the processor 210 from the extraluminal imaging device 215 may comprise metadata. The metadata of the contrast-agent angiographic images 220 may be, for example, DICOM metadata and may comprise additional information about the image data, such as the size, dimensions, bit depth, modality used to create the data, and equipment settings used to capture the image. The metadata may be used in the reconstruction of the blood vessel's geometry and generating of the reconstructed geometry object 545.

When executing the method 500, the geometry of the blood vessel 110 may be generated for the first state of the blood vessel 110. For example, if the execution of the method 500 starts with the “rest, pre-PCI” state, the reconstructed geometry object 545 is generated at step 540. When the preliminary routine 505 is executed for the subsequent state(s) of the blood vessel 110 (for example, “hyperemia, pre-PCI”), the reconstructed geometry object 545 generated for the first state of the blood vessel 110 (“rest, pre-PCI”) may be re-used for subsequent registration with the values of the blood pressure 225 and pressure-measurement angiographic images 222 at step 530.

Based on the contrast-agent angiographic images 220, the blood flow (Q) may be estimated. As illustrated in FIG. 5A, after the segmentation step 520, the processor 210 estimates the blood flow at step 525. The blood flow may be estimated based on at least one view. In at least one embodiment, the blood flow may be estimated for the whole coronary tree. Alternatively, the processor 210 may estimate a blood flow field spatially distributed in the coronary tree. In some embodiments, the blood flow (Q) may be estimated using a method described in the specification of U.S. Pat. No. 11,369,277.

In at least one embodiment, the flow is estimated by the flow routine 525 using the reconstructed geometry object 545. The reconstructed geometry object 545 may be calculated during the reconstruction (for example, a three-dimensional reconstruction) at the geometry reconstruction routine implemented at step 540 of method 500. The flow may be alternatively estimated by assuming a 2D axisymmetric geometry of the blood vessel 110. In other words, to estimate the flow, the angiographic images may be used in an axisymmetric model of the blood vessel, assuming that the vessel is located on a plane (flat) surface.

The value of the blood flow generated based on the contrast-agent angiographic images 220 or the reconstructed geometry object 545 may be used in a physical model routine 550 and in an optimization routine 570 of the method 500.

In at least one embodiment, at step 530, data obtained from the contrast-agent angiography contrast images 220 on one hand, and data obtained from the pressure-measurement angiographic images 222 and simultaneous pressure measurements 527, 529 on the other hand, may be merged.

Based on the pressure-measurement angiographic images 222, the processor 210 at step 530 may determine and store a position of the distal tip 315 of the pressure guidewire 305 in the blood vessel 110 and obtain a sequence of locations of the endoluminal data-acquisition device 300 as a function of time and two coordinates (x, y). For example, the location of the distal pressure sensor 310 at a certain time may be represented by coordinates (x, y). The pressure measurements 527, 529 may be averaged or dynamic. At each time step, the pressure-measurement angiographic images 222 may comprise one or more angiographic images taken (acquired) at one or more geometric planes and thus provide one or more different views of the pressure guidewire 305 located inside the blood vessel 110. The pressure-measurement angiographic images 222 may thus be used to obtain registered pressure and geometry data 535.

In at least one embodiment, the measured proximal pressure 529 and distal pressure 527, or distal pressure 527 alone, may be superimposed with (mapped onto) the reconstructed geometry object 545 of the blood vessel 110. For example, the reconstructed geometry object 545 may be obtained at the end of the diastole phase, and used for the superimposition. Thus, at step 530, the processor 210 may overlay the pressure measurements with the geometry of the blood vessel 110 determined (obtained) earlier. Alternatively, the processor 210 may map the pressure measurements 527, 529 with respect to a location of the distal pressure sensor 310 on the pressure-measurement angiographic image 222. Such steps may be also referred to as “registration” or “co-registration” of the pressure measurements to the coordinates of the blood vessel 110. Registration or co-registration as used herein refers to transforming different sets of data into one coordinate system.

Yet in another embodiment, alternatively, or in addition to mapping onto the reconstructed geometry object 545, the pressure values 527, 529, received as a pressure signal from the endoluminal data-acquisition device 300, may be superimposed with (mapped onto) the contrast-agent angiographic images 220 (acquired at the contrast-agent image acquisition step 510). To do so, in at least one embodiment, the processor 210 may calculate median pressure, at the end of diastole, and use the pressure-measurement angiographic image 222 taken (acquired) at the end of the diastole phase (which corresponds to the relaxed phase of the cardiac cycle). In at least one embodiment, the processor 210 may average the values of the blood pressure 527,529 measured over the time.

In at least one embodiment, the measured proximal pressure Pa and distal pressure Pd (or distal pressure 527 alone) and pressure-measurement angiographic images 222 obtained simultaneously with the pressure measurements 527, 529 (or the distal pressure 529 alone) may be mapped together onto the reconstructed geometry object 545 of the blood vessel 110 that has been obtained earlier at step 540, where the geometry of the blood vessel 110 has been determined based on the contrast-agent angiographic images 220 obtained for different planes (views) with the contrast agent in the blood vessel 110.

In at least one embodiment, to “register” the pressure measurements at step 530, the pressure measurements 527, 529 (or distal pressure 527 alone) may be first superimposed with the pressure-measurement angiographic image 222 to obtain a pressure-registered data comprising one or more images illustrating tracking of displacement of the endoluminal data acquisition device 300 inside the blood vessel 110. Then, such pressure-registered data may be superimposed with one of the contrast-agent angiographic image 220 or, preferably, with the reconstructed geometry object 545 to obtain registered pressure and geometry data 535. The resulting superimposed image, obtained at step 530, may be the displayed on the display 240. The distal pressure 527 may be registered with the pressure-measurement angiographic images 222, and then the pressure-measurement angiographic images 222 may be registered with the reconstructed geometry object 545.

Referring again to FIG. 5A, the flow data (such as the flow) determined at step 525, the registered pressure and geometry data 535, and, in some embodiments, the reconstructed geometry object 545 determined at step 540, are then transmitted to a physical model routine 550.

The physical model routine 550 (also referred to herein as a “step 550”) implements (applies) a physical model of blood distribution (flow) in the blood vessel 110. The physical model routine 550 uses the flow data (estimated at step 525), reconstructed geometry object 545 (in other words, reconstructed 3D (or 2D) parameters of the blood vessel 110 obtained from contrast-agent angiographic images 220), and the distribution of the blood pressure within the blood vessel (in other words, along the blood vessel 110) obtained by pressure measurements 512.

The physical model may be implemented using a CFD analysis. The physical model routine 550 solves Navier-Stokes equations which are partial differential equations describing the motion of blood within the blood vessel 110. When implemented in 0D, the physical model routine 550 may solve differential equations. In some embodiments, the physical model routine 550 may be executed in steady state (without dependence on time) using algebraic equations. The physical model may be data driven as in a reduced order model.

The physical model is based on the geometry of the blood vessel 110 (such as the reconstructed geometry object 545), boundary conditions (inflow boundary condition such as flow) and invasive (intra-vessel) measurements of pressure and flow. The physical model may solve Navier-Stokes equations either in three-dimensions (3D), two-dimensions (2D), one dimension (1D), or zero dimensions (0D). Yet another alternative physical model may be based on machine learning. In at least one embodiment, the physical model routine 550 uses the reduced order model or machine learning in order to solve the Navier-Stokes equations. The CFD, reduced order model and/or machine learning calculations may use the database 230 which may store initial values for implementation of the physical model.

The output of the physical model and therefore the output of step 550 of the method 500 is a set of output parameters 555 which comprises pressure estimates (which may be also referred to as “predictions” or “modified values of the blood pressure” or “solutions of the model”) relative to the coordinates and flow as a function of coordinates. Optimization implemented by the optimization routine 570 helps to find microvascular resistance. In some embodiments, the output parameters 555 of the physical model may comprise, for example, pressure estimates along a centerline of the blood vessel 110. In other words, the output of the physical model may be coordinates of an imaginary line running through a geometrical center of the blood vessel 110 and pressure estimates along that imaginary line as determined by the physical model. Thus, the output of the physical model of step 550 is the determined distribution of the blood pressure with regards to the space coordinates and therefore with regards to the blood vessel.

The physical model at step 550 uses the pressure and geometry data 535 received from the registration step 530 to generate a set of output parameters that comprises calculated (determined) pressure values in the volume of the blood vessel 110. After solving the Navier-Stokes equations, the physical model routine 550 provides the set of output parameters 555 to the optimization routine 570. The pressure values calculated at step 550 are then used in an objective function at the optimization routine 570 described below.

In at least one embodiment of method 500, the pressure measurements in the blood vessel 110 are performed by the guidewire 305 and the proximal pressure sensor 312 of the catheter 307 as described above for step 510, and, at the same time, the determined (predicted) pressure (set of output parameters 555) may be obtained based on a fluid mechanics model with boundary conditions. The boundary conditions may be, for example, values of the blood pressure that were measured with the pressure sensor 312 at two locations of a portion (segment) of the blood vessel 110. Then, the data obtained by measurements at steps 512 and 515 (proximal pressure 529 and distal pressure 527) may be merged, at step 550, with the contrast-agent angiographic images 220 obtained at step 510 and Navier-Stokes equations may be solved. In at least one alternative embodiment, at step 530, the processor 210 may merge the reconstructed geometry object 545 (obtained based on the contrast-agent angiographic images 220) with data obtained at step 512 (pressure measurements 527, 529) and step 515 (pressure-measurement angiographic image 222).

The physical model 550 generates, as an output for a particular state, the set of output parameters 555 representative of both pressure predictions based on the contrast-agent angiographic images 220 and pressure measurement 222, 225 with respect to the reconstructed geometry object 545 (2D or 3D geometry) of the blood vessel 110. The output of the physical model 550 which is the set of output parameters 555 may be then used by the optimization routine 570.

Referring again to FIGS. 2, 5A and 5B, at a preliminary routine 505a, measurements, acquisition of the data, transmission to the processor 210 and calculations of the physical model for the first state of the blood vessel 110 are performed. Similar measurements, similar preliminary routines 505b, 505c, 505d may be executed for other states of the blood vessel 110 as described above: pre-PCI rest, pre-PCI hyperemic, post-PCI rest and post-PCI hyperemic.

After the physical model routine 550 has solved the differential equations, an optimization routine 570 of the physical model of the blood vessel 110 is implemented. The optimization routine 570 uses the set of output parameters 555, generated by the physical model(s) 550 based on measurements obtained during one or more states, and adjusts reconciliated pressure values based on a plurality of iterations at different states of the blood vessel 110.

Referring to FIG. 5B, for each state of the four states described above, the steps and the various routines of the preliminary routine 505 illustrated in FIG. 5A are implemented to determine the flow data and the output parameters 555 for each corresponding state (such as a first set of output parameters 555a). Each one of the preliminary routines 505a, 505b, 505c, 505d may provide pressure and geometry data 535, reconstructed geometry object 545 and estimated flow to the corresponding physical model routines 550a, 550b, 550c, 550d. Each one of the physical model routines 550a, 550b, 550c, 550d (also referred to herein collectively “physical model routines 550”) corresponding to one of the states may use the database 230 for the initial 3D model.

The first physical model routine 550a is configured to execute the first physical model, the second physical model routine 550b is configured to execute the second physical model, the third physical model routine 550c is configured to execute the second physical model, the fourth physical model routine 550d is configured to execute the fourth physical model. Any one or more of the first physical model, the second physical model, the third physical model, and/or the fourth physical model may be a three-dimensional (3D) physical model, a two-dimensional (2D) physical model, one-dimensional (1D) physical model, or a zero-dimensional (0D) physical model. Any one or more of the first physical model, the second physical model, the third physical model, and/or the fourth physical model may be a combination of physical models, also referred to herein as a hybrid physical model. Any one of the first, second, third or fourth physical models may be a machine-learning model.

The hybrid physical model may be, for example, a combination of a 3D physical model of a critical part of the coronary tree such as a lesion or bifurcation with a 0D physical model of healthy coronary arteries or of the microcirculation. Pressure and flow may be exchanged at the interface of the two types of models (3D and 0D models), potentially by converting flows into velocity profile based on Womersley's solutions or another hypothesized velocity profile. Alternatively, the hybrid physical model may be a combination of machine learning with a 0D modeling approach. The machine learning may be used in such a model to approximate the behavior of elements such as lesions or bifurcations in a model solved using a 0D solver.

The physical model routines 550a, 550b, 550c, 550d then provide one or more sets of output parameters 555 (of first, second, third, and fourth sets of output parameters 555a, 555b, 555c, 555d, respectively) to the optimization routine 570. In at least one embodiment, the optimization routine 570 receives registered pressure measurements on the reconstructed 2D or 3D geometry (reconstructed geometry object 545) from each one of the physical model routines 550a, 550b, 550c, 550d.

After the plurality of iterations, the optimization routine 570 generates the reconciliated pressure values as a function of coordinates (also referred to herein as “reconciliated pressure field”).

In at least one embodiment, the optimization routine 570 generates and/or adjusts one or more hemodynamic parameters such as, for example, the absolute microvascular resistance, and minimizes the difference between the pressure values predicted by the model based on the flow and contrast-agent angiographic images 220 on one side, and the measured blood pressure 225 without the contrast agent on the other side.

The optimization routine 570 may implement a data assimilation routine. In at least one embodiment, the data assimilation routine comprises minimizing an objective function by weighting data correction with weights based on uncertainties. The weights may be pre-determined and correspond to the pre-determined accuracy of the values of the predicted (calculated) pressure and measured pressure. The objective function may be, for example: a difference between predicted and measured pressures and/or flows, and the data assimilation routine may thus minimize such a difference. For example, the data assimilation may comprise minimizing several objectives. In some embodiments, the weights may be uniform (for example, be equal to 1) and the data assimilation may comprise minimizing an error between the predicted pressure and the measured pressure.

When the input data is dynamic, which is when the input data to the algorithm is pressure P(t) and flow Q(t), then assimilation may be done by filtering. For example, the optimization routine 570 which implements the data assimilation may use an ensemble Kalman filter.

The optimization steps may depend on time, subsequent measurement in one state and/or the state of blood vessel 110. At the later optimization steps, each current set of output parameters 555 received from the physical model is compared with previously received set of output parameters 555, and the processor 210 adjusts pressure values, geometry values and/or boundary conditions. In at least one embodiment, the processor 210 adjusts first the boundary conditions, such as, for example, microvascular resistance R_{M} or IMR. Alternatively, a steady-state optimization may be performed after all the measurements have been performed. The implementation of a model with dynamic adjustment is more complex than implementation of the model in the steady state. In some embodiments, when implementing the dynamic adjustment, less assumptions are used, and the information is represented using signal dynamics. In at least one embodiment, the processor 210 performs the optimization over time or, alternatively, using averaged pressure values.

One or more blood vessel's states discussed above may be induced when the patient is on an examination table. The stent 120 may be installed, for example, after the first and the second states of the blood vessel 110, which correspond to the rest state and hyperemia state, have been implemented. Thus, after implementation of a first-state preliminary routine 505a and implementing the physical model routine 550a for each time step, the reconciled pressure is a value obtained based a combination of the data obtained with measurements and data obtained based on modeling (simulation). An initial value of the reconciled pressure may be one of the measured pressure or modeled pressure. Alternatively, the initial value of the reconciled pressure may be pre-defined (which may be referred to as a pre-defined initial reconciled pressure). The output of the physical model routine 550a permits adjusting the value of the reconciled pressure at each optimization step.

In operation of the method 500, the system 200 acquires a first set of contrast-agent angiographic images 220 and then, after the contrast-agent period of time, a first set of pressure-measurement angiographic images 222 and a first set of values of the blood pressure. The measurements are performed for the first state of the blood vessel 110: rest, pre-PCI state. A prompt to start a pullback of the endoluminal data-acquisition device 300 may be displayed on the display 240. The steps of the first preliminary routine 505a described above and the first physical model routine 550a may be implemented to generate the first set of output parameters 555a. In some embodiments, optimization may be performed for several time steps. In some embodiments, optimization may be performed by performing more than one pulling back of the endoluminal data-acquisition device 300, for example.

To improve the accuracy of the reconciled pressure with the optimization routine 570, the measurements at the second state (hyperemia pre-PCI state) of the blood vessel 110 may be performed. The operator may be then requested by a prompt on the display 240 to induce the hyperemic state of the blood vessel 110 and/or to confirm (for example, by pushing a button or by pushing a pre-determined portion of the display 240) that the second state has been induced. In some embodiments, the hyperemia may be detected from the pressure signal. To obtain a second set of contrast-agent angiographic images 220 in the second state of the blood vessel 110, introduction of the contrast agent and the introduction of the hyperemic agent are preferably close in time, as described above, in order to introduce the contrast agent simultaneously with the hyperemic state. Measurements of the second set of contrast-agent angiographic images 220 and then acquisition of the second set of pressure-measurement angiographic images 222 and the second set of values of the blood pressure 225 with stress may be started and may continue for example, until convergence of the results. In some embodiments, the second state may be requested to be induced first. The measurement data is therefore collected and the optimization routine 570 may be implemented first for the second state (hyperemia pre-PCI state). After implementation of the optimization routine 570 for one of the states, the optimized data may be displayed on the display 240.

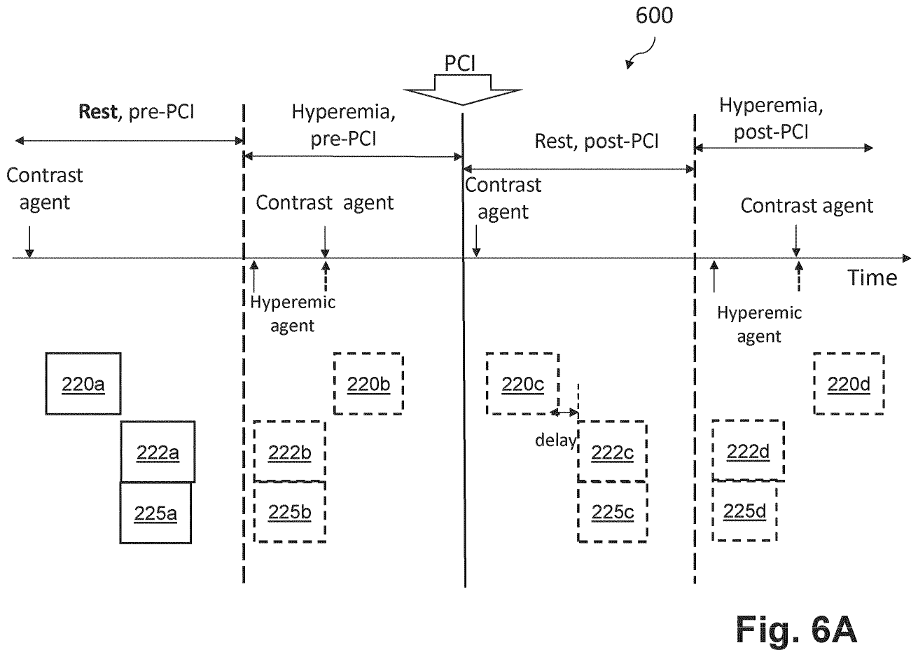

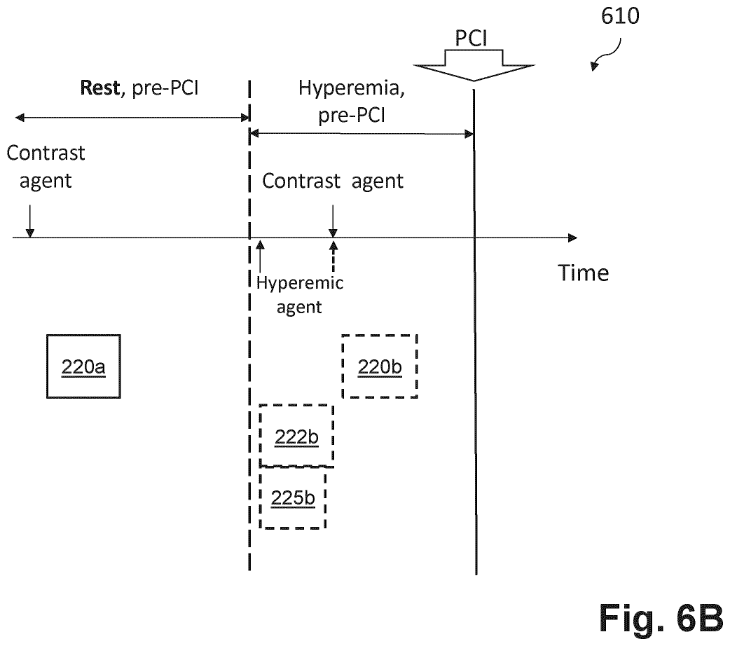

The optimization routine 570 may then request the user (the operator of the system 200 or the clinician) to induce another state of the four states—for example, install the stent and then confirm whether the third or the fourth state has been induced. The optimization routine therefore takes into account the data obtained during the previous optimization time periods and adjusts the optimization routine output based on the previous optimizations for the same blood vessels 110 and new measurements performed with the stent 120 (with or without hyperemia, i.e. third and fourth states). It should be noted that the stent 120 may be installed while the guidewire 305 is located inside the blood vessel 110. After the stent 120 is installed, optimizations in the third and fourth states may be implemented during the third and fourth optimization time periods, respectively. FIG. 6A schematically illustrates time diagram 600 of implementation of the method 500, in accordance with at least one embodiment of the present disclosure. As illustrated in FIG. 6A, preferably, the method 500 starts with measuring contrast-agent angiographic images 220. For each one of four states, the order of measurements of contrast-agent angiographic images 220 with reference to pressure-measurement angiographic images 222 and blood pressure 225 may vary and may be chosen by the user. For example, the user may provide information (input) regarding which measurements have been or are going to be executed. If the contrast-agent angiographic images 220 is measured first, the user may preferably need to wait (for example, several seconds) after execution of contrast-agent angiographic images 220 and before acquiring pressure-measurement angiographic images 222 and blood pressure 225, to let the blood pressure to stabilize after the introduction of the contrast agent. In other terms, there is a preferable delay between the introduction of the contrast agent (and therefore acquiring contrast-agent angiographic images 220) and the acquisition of pressure-measurement angiographic images 222 and blood pressure 225.

With reference to FIGS. 5A and 6A, when the acquisition of measurements data is performed for more than one state of the blood vessel 110 (as illustrated in FIG. 6A), the reconstructed geometry object 545 may be generated once for the first state of the blood vessel 110 (for example, for rest, pre-PCI state). When executing subsequently the preliminary routine 505 of the method 500, the processor may use (re-use) the reconstructed geometry object 545 generated for the first state of the blood vessel 110 earlier based on the measurements of the contrast-agent angiographic images 220 done for the first state of the blood vessel 110.

FIG. 6B schematically illustrates another time diagram 610 of implementation of the method 500 for determining hemodynamic parameters, in accordance with at least one embodiment of the present disclosure. As illustrated in FIG. 6B, the contrast-agent angiographic images 220 may be measured after the contrast agent has been introduced into the blood vessel 110 when the blood vessel 110 is in rest and may be subsequently used to generate the geometry of the blood vessel (step 540 at FIG. 5A) and to estimate the flow. The measurements of the contrast-agent angiographic images 220 may be followed by introducing the hyperemic agent and measuring pressure-measurement angiographic images 222 and blood pressure 225 when the blood vessel 110 is in hyperemia. Subsequent measurement of contrast-agent angiographic images 220 may be used to re-estimate the flow in the hyperemia state and to adjust the reconciled pressure at step 580 (FIG. 5B).

In at least one embodiment, if the measurements have been executed and registered during the hyperemia (second state or fourth state), the equation system may be improved by additional equations considering a state where the geometry of the blood vessel 110 has not been changed, while the myocardial resistance has been changed. In addition, the system 200 may determine the IMR value during the hyperemia. The system 200 may determine microvascular resistance R_{M} during the rest state and/or during hyperemia of the blood vessel 110. It at least one embodiment, several states of hyperemia may be used to generate additional equations. For example, measurements may be executed and registered during a rest state, during an intermediate hyperemia state caused by the contrast agent, and during a full hyperemia state caused by a hyperemic agent such as, for example, adenosine.

In at least one embodiment, for the measurements executed and data (such as corresponding angiographic images and pressure measurement data) registered in the post-PCI hyperemic state (fourth state) of the blood vessel 110, the equation system at the optimization routine 570 may have additional equations. These additional equations take into account the state where the lesion model has changed for example, due to PCI, but the myocardial resistance has not. In other terms, the presence of the stent 120 changes the geometry of the coronary artery, but does not change the microvascular resistance. Taking into account the post-PCI hyperemic state may help to improve precision of the reconciliated pressure which is generated by the optimization routine 570.

In at least one embodiment, the optimization routine 570 takes into account the fact that the blood vessel 110 may have two types of resistances: the resistance caused by the lesion and the microvascular resistance. Thus, for the first state of the blood vessel 110, a first blood flow Q₁ (determined, for example, at step 525) corresponds to a rest resistance R₁ of the lesion (stenosis 1 in FIG. 1C) and rest microvascular resistance R_{M-r} (which may be both determined based on the first set of output parameters 555a, obtained at the first state, that are received from the corresponding first physical model 505a). The first set of output parameters 555ₐ—Pₐ₁—obtained for the first state is proportional to Q₁(R₁+R_{M}): Pₐ₁˜Q₁(R₁+R_{M-r}).

For the pre-PCI hyperemia (second) state, the second set of output parameters Pₐ₂ is proportion to a second blood flow Q₂ and to a sum of a hyperemia resistance R₂ of the lesion and hyperemia microvascular resistance Rₘ₋ₕ: Pₐ₂˜Q₂(R₂+R_{M-h}).

The hyperemia resistance R₂ and rest resistance R₁ are related to each other via geometry of the blood vessel 110 and provided by reconstructed geometry object 545 in each one of the states. In at least one embodiment, the optimization routine 570 uses the reconstructed geometry objects 545 of the blood vessel 110 generated separately at rest and at hyperemia (the first and the second states), as well as the first and second sets of output parameters 555a, 555b generated by the physical models 550 for the first and the second states. By using the above equations, the optimization routine 570 adjusts the reconciled pressure.

In the third state—rest post-PCI state of the blood vessel 110, the third set of output parameters 555c is a function of (or is related to) a third blood flow Q₃ and rest microvascular resistance R_{M-r} and may be represented as: Pₐ₃˜Q₃(R_{M-r}). In the fourth state—hyperemia post-PCI state—the fourth set of output parameters 555d is a function of (or proportional to) a fourth blood flow Q₄ and the rest microvascular resistance R_{M-h}: Pₐ₄˜Q₄(R_{M-h}). The equations provided herein assume that the resistance after PCI is neglectable. In at least one embodiment, the model may include such a resistance. The optimization routine 570 may take into account the available third and/or the fourth sets of output parameters 555c, 555d and the above equations in order to determine the values of the reconciled pressure along the blood vessel 110. Similar equations may be determined when more than one lesion (stenosis area) is present in the blood vessel 110, and the optimization routine 570 may take into account that one lesion has the stent 120, and the other lesion(s) do(es) not have the stent 120. For example, the equations used by the optimization routine 570 after the introduction of the stent 120, may comprise, in addition to R_{M}, other lesions' resistances in the same blood vessel 110.

In order to determine the values of the reconciled pressure, the optimization routine 570 thus takes into account the available sets of output parameters 555 for each one of the states of the blood vessel 110 and their relation to the blood flow at the corresponding states. The optimization routine 570 also takes into account the reconstructed geometry object 545 determined at the corresponding states. By adjusting the reconciled pressure using the optimization routine 570, the values of the reconciled pressure may become more accurate with each optimization step. The optimization routine 570 implemented by the processor 210 generates the distribution of the reconciled pressure within the blood vessel (in other terms, a pressure field with regard to the coordinates, Prec(x, y, z, t)), flow and the reconciled microvascular resistance. Based on this output, the processor 210 may then generate the reconciled hemodynamic parameter at step 580 and display the reconciled hemodynamic parameter 270 on the display 240 at step 585. The reconciled hemodynamic parameter 270 may be, for example, IMR, FFR, a coronary flow reserve (CFR), diastolic pressure ratio (dPR), an absolute flow, an absolute resistance for either one or many coronary branches, and/or a ratio of absolute resistance during rest and/or hyperemia. In one embodiment, the pressure measurements 527 and 529, obtained during a pullback or point measurement, may be located using the position of the tip of the guidewire 305 in the angiographic images corresponding to measured pressure (pressure-measurement angiographic images 222) and registered (at step 530) on a 2D geometry of the vessels (reconstructed geometry object 545) or on a region of interest of the vessels derived from one single angiographic view with contrast agent (step 510). The angiographic view may be, for example, a single image taken at the end of the diastolic phase. The model may be a 0D cardiovascular model of the artery of interest. The model may use the measured pressures or flows as boundary conditions. Based on a single state (505a, 505b, 505c, or 505d) or multiple states, the pressure measurements may be assimilated with the model predictions using, for example a weighted average. The weights may be based on the confidence associated to the measurements and the predicted values. The reconciled hemodynamic parameters, for example, dPR, FFR, or reconciled pressures, may be displayed on a reference image, such as the vessel geometry (reconstructed geometry object 545) or reference angiography (for example, one of the contrast-agent angiographic images 220) using, for example, a symbols overlay to represent the reconciled pressure drops (such as, for example, a difference between values of the reconciled pressure for two points along the vessel) or gradients thereof, a color overlay showing the values of the reconciled pressure along the blood vessel(s) of interest, and/or a values overlay displaying the value(s) of the reconciled hemodynamic parameter(s) on the blood vessels. The values of the reconciled hemodynamic parameters may be used to determine features on the reference image to be displayed in derived views. For example, based on such determination of the feature, the display 240 may present to the user a graph showing the value(s) of the reconciled hemodynamic parameter(s) as a function of position on the vessel geometry or the reference angiography. Based on this displayed graph, the user may determine (choose) the length of a stent to be deployed in the blood vessel.

In at least one embodiment, the optimization routine 570 modifies one or more parameters that is (or are) common (also referred to herein as “common parameter(s)”) to two or more physical models 550, corresponding to the states, that are used for optimization by the optimization routine 570. When measurements at more than one state are used for the optimization, and therefore the output from several physical models 550 are used at optimization at step 570, then the common parameter(s) may be shared among the optimization routine 570 and at least two physical models 550a, 550b, 550c, 550d, each corresponding to one state. The common parameter(s) may be, for example, boundary conditions and/or geometry of the blood vessel 110. The optimization routine 570 may have one objective function for the output of all physical models corresponding to the states of the blood vessel 110 used for the optimization. For example, when any one of the common parameter(s) changes, it may be shared among two or all physical models. The error may be then minimized for the objective function taking into account all the output 555 of all physical models 550 involved, with the shared common parameter(s) used in the execution of the physical models 550. In at least one embodiment, the method 500 may comprise sharing at least one common parameter among at least two physical models when executing the physical models for two or more states of the blood vessel. The first, second, third, and fourth physical models 550a, 550b, 550c, 550d may be part of (may form) one coupled (common) physical model that has one common equation system.

For example, the CFR may be calculated based on the reconciled pressure obtained for at least two states: hyperemia and rest, pre-PCI. The IMR may be calculated based on the calculated blood flow. In at least one embodiment, an absolute resistance may be calculated at step 580 based on the reconciled pressure. Diastolic pressure ratio (dPR) may be defined as a ratio of the mean distal pressure P_{d} to the mean aortic pressure Pₐ during diastole at rest. FFR may be determined as a ratio of distal pressure (P_{d}) to proximal pressure (Pₐ) in hyperemia. The absolute flow may be determined as an input flow into the coronary artery or in any of the branches, expressed in ml/s. The absolute resistance may be determined as a pressure drop caused by a segment of the blood vessel 110 for a given absolute flow, and is expressed in mmHg/ml/s.

In at least one embodiment, a reconciliated pressure field is also generated at step 580. The reconciliated pressure field comprises values of reconciled pressure determined by the optimization routine 570 as a result of taking into account the output of one or several physical models 550a, 550b, 550c, 550d illustrated in FIG. 5B. To display the reconciliated pressure field, the processor 210 may generate a combined output image 275 (also referred to herein as “reconciliated pressure field image 275”). The combined output image 275 may be generated by superimposing values of reconciliated pressure (as determined by the optimization routine 570) with one of the angiogram images 220, 222 measured or with the reconstructed geometry object 545 (for example, the 3D modelled image) of the blood vessel 110. The combined output image 275 may have the same size/shape as the contrast-agent angiographic images 220 with the background 431 shown with black color (which may be referred to as “0” or “(0, 0, 0)” in RGB) and the blood vessel(s) 110. The blood vessels 110 may be shown with white color (which may correspond to “1” or “255”, depending on the number of bits) and various colors representing the values of the reconciliated pressure along the blood vessels. Various colors and/or color gradients and/or color codes may thus visually illustrate (visualize) values of the reconciliated pressure and the reconciliated pressure field relative to the geometry of the blood vessel(s) and the reconstructed geometry object 545. The colors and color codes may thus help the user to promptly identify visually a pressure drop in the blood vessel 110. The system 200 may also highlight the location of the pressure drop in the blood vessel 110 visually, on the combined output image 275, when displaying the combined output image 275.

FIG. 5B illustrates an example of the combined output image 275 in accordance with at least one embodiment. The background 431 of the combined output image 275 may be dark (for example, black) and the blood vessel(s) 110 may be illustrated with brighter colors, illustrating a variation or a gradient of colors in the blood vessel(s) that signals (illustrates) the value of the blood pressure along the blood vessel(s). An alternative example of the combined output image—inversed combined output image 276 is also illustrated in FIG. 5B, where colors of the combined output image 275 are inversed, illustrating the blood vessels with darker colors and the background with white color. In at least one embodiment, the combined output image 275 may be generated and the system may display a visual representation of the reconciliated pressure values superimposed with the reconstructed geometry object and representing a reconciliated pressure field.

One or more reconciled hemodynamic parameter(s), and/or the combined output image 275, are then rendered by the processor 210 to the display 240 and displayed to the user (operator, clinician) for decision-making. Based on the reconciled hemodynamic parameter(s) and/or combined output image 275, the clinician may assess the condition of the blood vessel 110 and decide whether any PCI and/or treatment is needed. For example, the clinician may decide whether any PCI is needed if the measurements have only been done prior to any PCI, or, alternatively, the clinician may judge whether the PCI has been successful, and that no additional intervention or other treatment is required.

The value of the reconciled hemodynamic parameter 270 may help to generate an indication of a patient condition, such as a microvascular obstruction (MVO) which characterizes a damage and dysfunction of the myocardial microvasculature. For example, the processor 210 may determine the severity of MVO based on calculated IMR values and display an indication of the rate of MVO or an indication that the patient has the MVO along with the calculated IMR. The reconciled pressure distribution field determined by the processor 210 may help to choose and suggest a location and a length of the stent that would permit to improve values of CFR and FFR. For this, the processor 210 may perform calculations using the reconciled pressure distribution and for a set of suggested locations and lengths of the stent 120.

Referring again to FIG. 5B, the optimization routine 570 may generate such output data as the reconciled pressure, the flow, the 3D geometry of the blood vessel (reconstructed geometry object 545) and boundary conditions and transmit them to the display routine 585. Inflow boundary condition may be, for example, the flow determined at the location of the proximal pressure sensor 312 and may be adjusted during the execution of the optimization routine 570. The outflow boundary condition may be, for example, the microvascular resistance and may be also adjusted by the optimization routine 570. While the display routine 585 is executed, the display 240 may present (display) a value of the reconciled hemodynamic parameter.

The method as described herein permits obtaining a representation of a field of pressure in the blood vessel 110 which permits determining location of the pressure drop in the blood vessel 110. Determining of the location of the pressure drop is difficult with currently known methods of measurement of the blood pressure because they do not permit to determine, with acceptable precision, where exactly the blood pressure has been measured in the blood vessel 110.

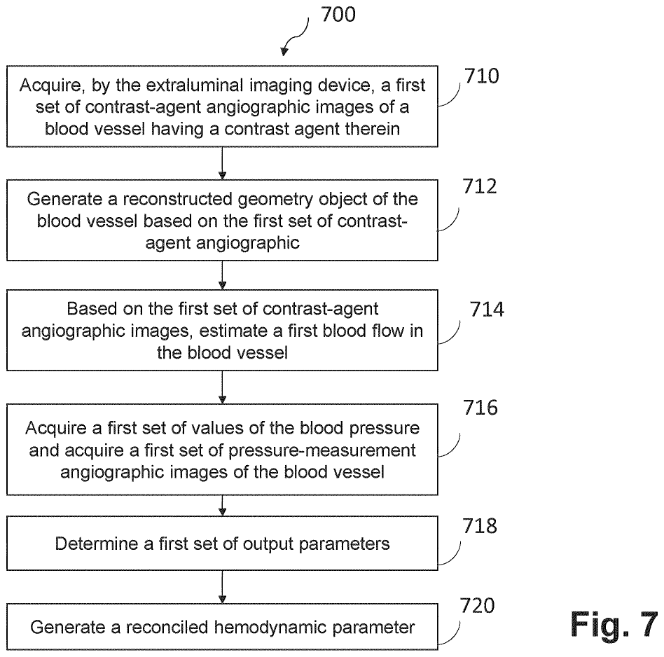

FIG. 7 illustrates the method 700 for determining hemodynamic parameters, in accordance with at least one embodiment of the present disclosure. Referring also to FIGS. 1D, 2, 5A, 5B and 6A, the method 700 of FIG. 7 is executable by the system 200 comprising the processor 210 in communication with an intravascular pressure measurement device 300 and with an extraluminal imaging device 215. At step 710, the extraluminal imaging device 215 acquires a first set of contrast-agent angiographic images 220a of a blood vessel 110 having a contrast agent therein. At step 712, a reconstructed geometry object 545 of the blood vessel 110 is generated based on the first set of contrast-agent angiographic images 220a. At step 715, the processor 210 estimates a first blood flow in the blood vessel 110 based on the first set of contrast-agent angiographic images 220a. At step 716, the intravascular pressure measurement device 300 acquires a first set of values of the blood pressure 225a as a function of a location within the blood vessel 110. The extraluminal imaging device 215 acquires a first set of pressure-measurement angiographic images 222a of the blood vessel 110.

At step 718, the processor 210 determines a first set of output parameters 555a based on the first set of values of the blood pressure 225a and based on the first blood flow. In at least one embodiment, the first set of values of the blood pressure 225a is registered with the first set of the pressure-measurement angiographic images 222a. The processor 210 implements a first physical model 550a of blood distribution in the blood vessel 110 using the reconstructed geometry object 545a. At step 720, the reconciled hemodynamic parameter is generated based on the first set of output parameters 555a.

The first set of contrast-agent angiographic images 220a, the first set of values of the blood pressure 225a and the first set of pressure-measurement angiographic images 222a may be obtained during a hyperemic state of the blood vessel 110, the blood vessel 110 having a hyperemic agent therein.

When the blood vessel 110 has a hyperemic agent therein, the extraluminal imaging device 215 may acquire a second set of contrast-agent angiographic images 220b of a blood vessel 100 having the contrast agent and hyperemic agent therein. Based on the second set of contrast-agent angiographic images 220b, the processor 210 may estimate a second blood flow in the blood vessel 110. The intravascular pressure measurement device 300 may acquire a second set of values of the blood pressure 225b as a function of location within the blood vessel 110 having the hyperemic agent therein, and a second set of pressure-measurement angiographic images 222b of the blood vessel 110 may be acquired with the extraluminal imaging device 215. A second set of output parameters 550b may be determined by the processor 210 based on the second blood flow and the second set of values of the blood pressure 225b, which may be registered with the second set of the pressure-measurement angiographic images 222b, by implementing a second physical model 550b of the blood distribution in the blood vessel 110 and using the reconstructed geometry object 545. The processor 210 may use the second set of the output parameters 555b when adjusting the reconciled hemodynamic parameter. The reconciled hemodynamic parameter may be adjusted based on the first set of the output parameters 555a and the second set of the output parameters 555b.

After the stent 120 has been installed or after another PCI in the blood vessel 110, a third set of contrast-agent angiographic images 220c of the blood vessel 110 having the contrast agent therein, the system may determine the third set of output parameters 555c based on the third set of values of the blood pressure 225c and the third blood flow determined based on the third set of contrast-agent angiographic images 220c acquired with the contrast agent in the blood vessel 110. In some embodiments, the third set of values of the blood pressure 225c may be registered with the third set of the pressure-measurement angiographic images 222c. The processor 210 may implement a third physical model 550c of the blood distribution in the blood vessel 110 using the reconstructed geometry object 545. The reconciled hemodynamic parameter may be further adjusted based on the third set of the output parameters 555c, in addition to the first and second sets of the output parameters 555a, 555b determined earlier.

The fourth state of the blood vessel 110, may be, for example, hyperemia post-PCI, as illustrated in FIG. 6A. Using a fourth physical model routine of the processor 210, a fourth set of output parameters 555d may be determined based on the fourth set of values of the blood pressure 225d, which may be registered with the fourth set of the pressure-measurement angiographic images 222d, and the fourth blood flow estimated from a fourth set of contrast-agent angiographic images 220 measured by the extraluminal imaging device 215 with the contrast agent. Using the geometry data 535 determined for the fourth state, the fourth physical model of the blood distribution in the blood vessel 110 may be implemented using the reconstructed geometry object 545 and the fourth blood flow. The reconciled hemodynamic parameter may be adjusted further based on the fourth set of the output parameters 555d.

While preferred embodiments have been described above and illustrated in the accompanying drawings, it will be evident to those skilled in the art that modifications may be made without departing from this disclosure. Such modifications are considered as possible variants comprised in the scope of the disclosure.

1-54. (canceled)

55. A method executable by a system comprising a processor in communication with an intravascular pressure measurement device and with an extraluminal imaging device, the method comprising:
acquiring, by the extraluminal imaging device, a first set of contrast-agent angiographic images of a blood vessel having a contrast agent therein;
generating a reconstructed geometry object of the blood vessel based on the first set of contrast-agent angiographic images;
based on the first set of contrast-agent angiographic images, estimating a first blood flow in the blood vessel;
acquiring, by the intravascular pressure measurement device, a first set of values of blood pressure and acquiring, by the extraluminal imaging device, a first set of pressure-measurement angiographic images of the blood vessel without the contrast agent;
registering the first set of values of the blood pressure with the reconstructed geometry object of the blood vessel;
determining, by the processor, a first set of output parameters based on the first set of values of the blood pressure and the first blood flow, by implementing a first physical model of blood distribution in the blood vessel using the reconstructed geometry object, the first blood flow and a first set of registered pressure measurements; and
generating by data assimilation a reconciled hemodynamic parameter based on the first set of output parameters.

56. The method of claim 55, wherein the first set of contrast-agent angiographic images and the first set of values of the blood pressure are obtained during a hyperemic state of the blood vessel, the blood vessel having a hyperemic agent therein.

57. The method of claim 55, wherein the first set of values of the blood pressure is a function of a location within the blood vessel.

58. The method of claim 55 further comprising determining the first set of output parameters, by the processor, based on the first set of values of the blood pressure registered with the first set of the pressure-measurement angiographic images.

59. The method of claim 55, wherein the method further comprises, when the blood vessel has a hyperemic agent therein:
acquiring, by the extraluminal imaging device, a second set of contrast-agent angiographic images of the blood vessel having the contrast agent and the hyperemic agent therein;
based on the second set of contrast-agent angiographic images, estimating a second blood flow in the blood vessel;
acquiring, by the intravascular pressure measurement device, a second set of values of the blood pressure within the blood vessel having the hyperemic agent therein; and
determining, by the processor, a second set of output parameters based on the second set of values of the blood pressure and the second blood flow, by implementing a second physical model of the blood distribution in the blood vessel and using the reconstructed geometry object;
wherein generating the reconciled hemodynamic parameter further comprises adjusting the reconciled hemodynamic parameter based on the first set of the output parameters and the second set of the output parameters.

60. The method of claim 59, wherein the second set of values of the blood pressure is a function of a location within the blood vessel.

61. The method of claim 59 further comprising acquiring, by the extraluminal imaging device, a second set of pressure-measurement angiographic images of the blood vessel and registering the second set of values of the blood pressure with the second set of the pressure-measurement angiographic images.

62. The method of claim 59 further comprising determining the second set of output parameters, by the processor, based on the second set of values of the blood pressure registered with the second set of the pressure-measurement angiographic images.

63. The method of claim 59, wherein the method further comprises:
acquiring, by the extraluminal imaging device, a third set of contrast-agent angiographic images of the blood vessel having a stent and the contrast agent therein;
based on the third set of contrast-agent angiographic images, estimating a third blood flow in the blood vessel;
acquiring, by the intravascular pressure measurement device, a third set of values of the blood pressure within the blood vessel; and
determining, by the processor, a third set of output parameters based on the third set of values of the blood pressure and the third blood flow, by implementing a third physical model of the blood distribution in the blood vessel using the reconstructed geometry object;
wherein the adjusting the reconciled hemodynamic parameter is further based on the third set of the output parameters.

64. The method of claim 55, wherein the acquiring of the first set of values of the blood pressure is simultaneous with the acquiring of the first set of pressure-measurement angiographic images of the blood vessel.

65. The method of claim 55, the method further comprising:
determining, by the processor, a second set of output parameters based on a second set of values of the blood pressure having a hyperemic agent therein and without the contrast agent, and a second blood flow determined from a second set of contrast-agent angiographic images acquired when the contrast agent and a hyperemic agent are in the blood vessel; and
wherein generating the reconciled hemodynamic parameter further comprises adjusting the reconciled hemodynamic parameter based on the first set of the output parameters and the second set of the output parameters.

66. The method of claim 65, further comprising acquiring a second set of pressure-measurement angiographic images of the blood vessel and registering the second set of values of the blood pressure with the second set of pressure-measurement angiographic images of the blood vessel.

67. The method of claim 65 further comprising, after a stent has been installed in the blood vessel, determining, by the processor, a third set of output parameters based on:
a third set of values of the blood pressure acquired with acquiring of a third set of pressure-measurement angiographic images after at least a third period of time elapsed since acquiring the second set of contrast-agent angiographic images and
a third blood flow determined from a third set of contrast-agent angiographic images acquired when the contrast agent is in the blood vessel; and
wherein adjusting the reconciled hemodynamic parameter is further based on the third set of output parameters.

68. The method of claim 55, wherein the reconciled hemodynamic parameter is at least one of an index of microvascular resistance, a fractional flow reserve, a coronary flow reserve, a diastolic pressure ratio, an absolute flow, an absolute resistance, and a ratio of absolute resistance.

69. A system comprising:
an extraluminal imaging device configured to generate a first set of contrast-agent angiographic images of a blood vessel having a contrast agent therein and a first set of pressure-measurement angiographic images acquired without the contrast agent in the blood vessel;
an intravascular pressure measurement device configured to measure a first set of values of blood pressure and generate endoluminal pressure data; and
a processor configured to:
generate a reconstructed geometry object of the blood vessel based on the first set of contrast-agent angiographic images;
based on the first set of contrast-agent angiographic images, estimate a first blood flow in the blood vessel;
register the first set of values of the blood pressure with the reconstructed geometry object of the blood vessel;
determine a first set of output parameters based on the first blood flow and the first set of values of the blood pressure, by implementing a first physical model of blood distribution in the blood vessel using the reconstructed geometry object, the first blood flow and a first set of registered pressure measurements; and
generate by data assimilation a reconciled hemodynamic parameter based on the first set of output parameters.

70. The system of claim 69, further comprising a display configured to display the reconciled hemodynamic parameter and an image representing a reconciliated pressure field.

71. The system of claim 69, wherein the first set of values of the blood pressure is registered with the first set of the pressure-measurement angiographic images.

72. The system of claim 69, wherein the processor is further configured to:
based on a second set of contrast-agent angiographic images acquired when the blood vessel had the contrast agent and hyperemic agent, estimate a second blood flow in the blood vessel;
determine a second set of output parameters based on a second set of values of the blood pressure acquired by the intravascular pressure measurement device, by implementing a second physical model of the blood distribution in the blood vessel and using the reconstructed geometry object; and
adjust the reconciled hemodynamic parameter based on the first set of the output parameters and the second set of the output parameters.

73. The system of claim 72, wherein the second set of values of the blood pressure is registered with a second set of pressure-measurement angiographic images acquired by the extraluminal imaging device.

74. A method executable by a system having a processor in communication with an extraluminal imaging device and an intravascular pressure measurement device, the method comprising:
acquiring contrast-agent angiographic images when the blood vessel has a contrast agent therein;
separately and for the same blood vessel and without the contrast agent, acquiring pressure-measurement angiographic images, acquiring intravascular pressure values;
reconstructing geometry of the blood vessel, registering a first set of values of blood pressure with a reconstructed geometry object of the blood vessel, and applying a physical model to the reconstructed geometry object; and
generating by data assimilation a reconciled hemodynamic parameter, and adjusting the reconciled hemodynamic parameter based on the contrast-agent angiographic images, pressure-measurement angiographic images, and intravascular pressure values acquired when the blood vessel is in at least two of blood vessel states selected from: rest prior to percutaneous coronary intervention (PCI), hyperemia prior to PCI, hyperemia post-PCI, and rest post-PCI.
